(19) United States
(12) Patent Application Publication
(10) Pub. No.: US 2003/0017124 A1
(43) Pub. Date: Jan. 23, 2003

(54) Two-coat make-up product containing a goniochromatic pigment and monochrome pigment, and make-up kit containing this product

(75) Inventors: Isabelle Agostini, Vemeres-le-Buisaon (FR); Sylvie Guillard, Chevry-Cossigny (FR); Pascal Arnaud, L'Hay Les Roses (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/119,010

(22) Filed: Apr. 10, 2002

(30) Foreign Application Priority Data
Apr. 10, 2001 (FR) 01 04939

(51) Int. Cl. A61K 7/021; A61K 7/025

(52) U.S. Cl. 424/63; 424/64

(57) ABSTRACT

The invention relates to a product, which contains two compositions, one composition containing polymer particles dispersed in a physiological medium and which is surface-stabilized with a stabilizer in a liquid phase and a first coloring agent; and a second composition containing a second coloring agent, one of the coloring agents being a goniochromatic agent and the other being a monochrome agent, and methods of using the product.

### CROSS-REFERENCE TO A RELATED APPLICATION

The present application claims priority to U.S. Provisional Application No. 60/239,979, which was filed on May 30, 2001.

### BACKGROUND OF THE INVENTION

1. Field of the Invention

The present invention relates to a novel cosmetic make-up product for the skin, the lips or integuments, combining at least one first goniochromatic pigment and at least one second pigment, especially a monochrome pigment.

This product comprises two compositions which may be applied successively to human skin either of the face or the body, to the lower and upper eyelids of human beings, to the lips and to integuments such as the nails, the eyebrows, the eyelashes or the hair, and also a two-coat make-up process for the human face and body.

Each composition may be a free or compacted powder, a foundation, a face powder, an eyeshadow, a concealer product, a blusher, a lipstick, a lip balm, a lip gloss, a lip pencil, an eye pencil, a mascara, an eyeliner, a nail varnish or a body make-up or skin-coloring product.

2. Discussion of the Background

Consumers are increasingly seeking to tailor and personalize their make-up. They are increasingly looking for novel make-up products containing coloring agents different than the ranges usually offered by cosmeticians.

Specifically, the range of coloring agents currently used by cosmeticians is relatively limited; these agents are mainly organic pigments, lakes, mineral pigments or nacreous pigments. The lakes make it possible to obtain vivid colors, that are for the most part unstable to light, temperature and pH. Some of them also have the drawback of leaving unsightly marks on the skin after application, due to running of the colorant. In contrast, mineral pigments, in particular mineral oxides, are very stable, but give colors that are rather dull and pale. As regards nacreous pigments, they make it possible to obtain colors that are varied, but never intense, with iridescent effects, but which are usually quite weak.

To satisfy this demand, the Applicant proposed in patent application EP-A-0 953 330 a novel make-up kit for the skin, the lips and/or integuments combining a first goniochromatic pigment and a second pigment having one of the colors of the first pigment, the goniochromatic pigment being capable of producing different colors depending on the incidence of the light and the angle of observation. This kit makes it possible to obtain a make-up result that changes according to the angle of observation and the incidence of the light.

However, the Applicant found itself confronted with the problem of migration and transfer of these products. The term “migration” means a running of the composition and in particular of the color beyond the initial line of the make-up. In addition, it was found that the make-up could have poor staying power over time, in particular of the color. This poor staying power is characterized by a modification of the color (color change or fading) generally following an interaction with the sebum and sweat secreted by the skin in the case of foundations and face powders, or of an interaction with saliva in the case of lipsticks. This obliges the user to apply fresh make-up very regularly, which may constitute wasted time.

Furthermore, these make-up products, especially for the lips and the skin, have a tendency to transfer, that is to say to become at least partially deposited on supports with which they come into contact (glass, clothing, cigarettes, fabrics, etc.).

To overcome the problems of transfer, cosmeticians have proposed compositions generally based on silicone resins and volatile silicone oils, which, although having improved properties as regards staying power, have the drawback of leaving on the lips and the skin, after the volatile silicone oils have evaporated off, a film that becomes uncomfortable over time (sensation of dryness and of tautness), which puts a certain number of women off this type of lipstick.

In addition, these compositions based on volatile silicone oils and silicone resins produce matte colored films. However, women are nowadays looking for products, especially for coloring the lips or the eyelids, that are glossy while at the same time having good staying power and being transfer-resistant.

Other companies have become interested in the problems of transfer, such as the company Kose which proposed in its patent application JP-A-05 221 829 the use of a gel based on perfluoro materials, which is applied over a film of lipstick so as to prevent it from transferring onto other surfaces, the gel being incompatible with the film of lipstick.

Although the use of perfluoro oils ensures incompatibility between the gel and the film of lipstick and thus staying power and transfer-resistance properties, formulations of this type have the drawback of having poor cosmetic properties, since the film of lipstick becomes oily and liable to migrate, which is unacceptable for consumers.

Mention may also be made of patent application WO-A-97/17057 from the company Procter & Gamble, which describes a method for increasing staying power and transfer-resistance properties, consisting in applying two compositions, one over the other. These two compositions satisfy the following physicochemical criteria:

global Hildebrand solubility parameters of less than 8.5 (cal/cm³)^{½} for the composition applied first,

presence of oil whose calculated partition coefficient ClogP is at least equal to 13 for the topcoat.

However, this composition selection does not exclude the possibility of having the same constituents in the two compositions. Specifically, triglycerides, in particular sweet almond oil and olive oil, mentioned as satisfying the partition coefficient criteria, also have Hildebrand solubility parameters of less than 8.5 (cal/cm³)^{½} (Vaughan C. D. “Solubility effects in product, package, penetration and preservation”, Cosmetics and Toiletries, vol. 103, pp. 47-69, 1988):

Sweet almond oil: 6.81 (cal/cm³)^{½}.

Olive oil 7.87 (cal/cm³)^{½}.

Consequently, there is a certain level of compatibility between the two coats, which does not make it possible to have entirely satisfactory staying power and transfer-resistance properties.

Finally, patent U.S. Pat. No. 6,001,374 from Nichols proposes a multilayer make-up system that consists in using a composition containing an alcohol-soluble and water-insoluble resin, which may be applied as a basecoat or as a topcoat, and which has the advantage of not leaving marks on a support placed in contact with the make-up, and of being resistant to water and to friction, while at the same time having a certain level of gloss. However, this composition contains a water-soluble alcohol, in particular ethanol, which is a compound that has an irritant, dehydrating nature on the skin and more particularly on the lips, and which is particularly uncomfortable when the skin or the lips are damaged. Furthermore, this composition requires the use of a particular make-up remover, which is not particularly practical.

### SUMMARY OF THE INVENTION

The aim of the present invention is to propose a make-up product that combines at least one first goniochromatic pigment and at least one second monochrome pigment, which, while allowing a novel make-up to be obtained, simultaneously combines the properties of “transfer resistance”, migration resistance, staying power, comfort, absence of dehydration, and gloss, this result not having been satisfactorily obtained hitherto.

The Applicant has found, surprisingly, that by combining a first composition comprising a first physiologically acceptable medium containing polymer particles dispersed and surface-stabilized by means of a stabilizer in a liquid phase and at least one first coloring agent and a second composition comprising, in a second physiologically acceptable medium, at least one second coloring agent, one of the coloring agents being a goniochromatic agent and the other being a monochrome agent, a glossy two-coat make-up is obtained, which does not migrate, does not transfer, has good staying power and is not greasy, while at the same time being comfortable when applied and over time (does not dehydrate the skin or make it taut).

Furthermore, it has been found that the product according to the invention has particularly advantageous qualities of spreading on and adhesion to the skin, the lips, the eyelashes or mucous membranes, and also has a pleasant, creamy feel. The product also has the advantage of being easy to remove, especially with a standard make-up remover.

### DETAILED DESCRIPTION OF THE INVENTION

The expression “goniochromatic coloring agent” means a coloring agent whose colors vary according to the angle of observation and the incidence of the light, and which gives iridescent effects a little like a nacreous product.

In particular, the product of the invention allows the production of continuous deposits that are not sticky, with good coverage having a glossy appearance, adapted to the consumer's desire, that are migration-resistant, have good staying power and do not dehydrate the skin or the lips onto which it is applied, either during application or over time. It also has good stability properties and gives an attractive, uniform make-up result.

These properties of staying power, transfer resistance and migration resistance, combined with the glossy, comfortable and non-greasy appearance, make a product particularly suitable for producing make-up products for the lips such as lipsticks, or lip glosses, or for the eyes, such as mascaras, eyeliners and eyeshadows.

One subject of the present invention is thus a cosmetic make-up product containing a first and a second composition, the first composition comprising, in a first physiologically acceptable medium, polymer particles dispersed and surface-stabilized by means of a stabilizer in a liquid phase and at least one first coloring agent, and the second composition comprising, in a second physiologically acceptable medium, at least one second coloring agent, one of the coloring agents being a goniochromatic agent capable of producing different colors depending on the incidence of the light and the angle of observation, the other being a monochrome agent.

The expression “make-up product” means a product containing a coloring agent allowing a color to be deposited onto a keratin material (the skin, the lips or integuments) of a human being, such as a lipstick, a face powder, an eyeliner, a foundation, a self-tanning product or a semipermanent make-up product (tattoo).

The product according to the invention comprises two (or more) physiologically acceptable compositions packaged separately or together in the same packaging article or in two (or more) separate or distinct packaging articles.

Preferably, these compositions are packaged separately and, advantageously, in separate or distinct packaging articles.

One subject of the present invention is thus, in particular, a cosmetic make-up product in the form of a foundation, a face powder, an eyeshadow, a lipstick, a colored make-up product especially having lipcare properties, an eyeliner, a concealer product or a body make-up product (of the tattoo type).

A subject of the invention is also a make-up kit containing a cosmetic make-up product as defined above, in which the various compositions are packaged separately and are accompanied by suitable application means. These means may be fine brushes, coarse brushes, pens, pencils, felts, nibs, sponges and/or foams. Felts are preferably used.

The first composition in the product according to the invention can constitute a basecoat applied to the keratin material, and the second composition a topcoat. However, it is possible to apply, under the first coat, an undercoat which may or may not have the constitution of the second composition. It is also possible to apply an overcoat onto the second coat, which may or may not have an identical constitution to that of the first coat.

Preferably, the make-up obtained is a two-coat make-up.

In particular, the basecoat is a foundation, a face powder, a lipstick, a lip gloss, an eyeliner or a body make-up product, and the topcoat is a protective or care product.

The invention also relates to a make-up process for the skin and/or the lips and/or integuments, which consists in applying a cosmetic make-up product as defined above to the skin and/or the lips and/or integuments.

A subject of the invention is also a make-up process for the skin and/or the lips and/or integuments, which consists in applying to the skin, the lips and/or integuments a first coat of a first composition comprising, in a first physiologically acceptable medium, polymer particles dispersed and surface-stabilized by means of a stabilizer in a liquid phase and at least one first coloring agent, and then in applying, over all or some of the first coat, a second coat of a second composition comprising, in a second physiologically acceptable medium, at least one second coloring agent, one of the coloring agents being a goniochromatic agent capable of producing different colors depending on the incidence of the light and the angle of observation, the other agent being a monochrome agent.

Preferably, the first coat of the first composition is allowed to dry before applying the second coat of the second composition.

When the topcoat contains a monochrome agent having one of the colors of the goniochromatic agent, it is possible to apply the second coat over only a portion of the first coat.

When the second coat contains a monochrome agent having one of the colors of the goniochromatic agent, it is possible to mark out or draw patterns on such a coat (letters, designs, checks, etc.), in particular with a pencil or fine brush and, at a certain angle of observation, especially perpendicular to the second coat, the patterns of this second coat will disappear because their color will be identical to that of the goniochromatic coat, and in the other directions, the patterns will appear because they are of a different color than that of the first coat.

This two-coat make-up may be adapted to all make-up products for the skin, not only for the face but also for the scalp and the body of human beings, mucous membranes, for instance the lips and the inner edge of the lower eyelids, and integuments, for instance the nails, the eyelashes, the hair, the eyebrows, or even body hairs. The second coat can form patterns, and can be applied with a pen, a pencil or any other instrument (sponge, finger, fine brush, coarse brush, feather, etc.). This make-up may also be applied to make-up accessories, for instance false nails, false eyelashes, wigs or small or large patches adhering to the skin or the lips (of the beauty-spot type).

A subject of the invention is also a cosmetic composition for carrying out the make-up process described above. This composition comprises, in a physiologically acceptable medium, polymer particles dispersed and surface-stabilized by means of a stabilizer in a liquid phase, a coloring agent chosen from goniochromatic agents capable of producing different colors depending on the incidence of the light and the angle of observation, or monochrome agents and a rheological agent chosen from olefin copolymers of controlled crystallization, and mixtures thereof. The rheological agent is preferably an ethylene/octene copolymer.

A subject of the invention is also a made-up support comprising a first coat of a first composition comprising, in a first physiologically acceptable medium, polymer particles dispersed and surface-stabilized by means of a stabilizer in a liquid phase and at least one first coloring agent, and a second coat of a second composition, applied over all or some of the first coat, comprising, in a second physiologically acceptable medium, at least one second coloring agent, one of the coloring agents being a goniochromatic agent capable of producing different colors depending on the incidence of the light and the angle of observation, the other agent being a monochrome agent, the second composition being in solid form.

This support may in particular be a hairpiece such as a wig, false nails, false eyelashes or patches adhering to the skin or the lips (of the beauty-spot type).

The invention also relates to the use of a cosmetic make-up product defined above for improving the comfort and/or gloss and/or transfer and/or migration properties of the make-up, and/or the staying power on the skin and/or the lips and/or integuments of human beings.

First Composition

The first composition according to the invention thus comprises, in a first physiologically acceptable medium, polymer particles dispersed and surface-stabilized by means of a stabilizer in a liquid phase (referred to hereinbelow as “polymer dispersion”) and at least one first coloring agent.

The expression “physiologically acceptable medium” means a nontoxic medium that may be applied to the skin, integuments or the lips of the face of human beings.

Polymer in Dispersion

According to the invention, the polymer is a solid that is insoluble in the liquid phase of the first composition even at its softening point, unlike a wax even of polymeric origin, which is soluble in a liquid organic phase (or fatty phase) at its melting point. It also allows the formation of a deposit, especially a homogeneous, continuous, film-forming deposit, and/or is characterized by the entanglement of the polymer chains. With a wax, even one obtained by polymerization, a recrystallization is obtained after melting in the liquid organic phase. This recrystallization is in particular responsible for the loss of gloss of the composition.

In order to have optimum transfer-resistance properties, the amount of polymer is chosen as a function of the amount of dyestuffs and/or active agents and/or oils contained in the first composition. In practice, the amount of polymer may be greater than 2% by weight (of active material) relative to the total weight of the composition.

One advantage of using a dispersion of polymer particles in a composition of the invention is that these particles remain in the form of elementary particles, without forming aggregates, in the liquid phase. Another advantage of the polymer dispersion is the possibility of obtaining very fluid compositions (of the order of 130 centipoises), even in the presence of a high content of polymer.

Yet another advantage of such a polymer dispersion is that it is possible to calibrate as desired the size of the polymer particles, and to modify their size “polydispersity” during the synthesis. It is thus possible to obtain particles of very small size, which are invisible to the naked eye when they are in the composition and when they are applied to the skin, the lips or integuments.

Another advantage of the polymer dispersion of the composition of the invention is the possibility of varying the glass transition temperature (Tg) of the polymer or of the polymer system (polymer plus additive of the plasticizer type), and thus to go from a hard polymer to a more or less soft polymer, allowing the mechanical properties of the composition to be adjusted as a function of the intended application and in particular of the film applied.

The first composition of the product according to the invention thus advantageously comprises at least one stable dispersion of generally spherical polymer particles of one or more polymers, in a physiologically acceptable liquid phase. These dispersions may especially be in the form of polymer nanoparticles in stable dispersion in said liquid phase. The nanoparticles preferably have a mean size of between 5 and 800 nm and better still between 50 and 500 nm. However, it is possible to obtain polymer particles ranging up to 1 μm in size.

Preferably, the polymer particles in dispersion are insoluble in water-soluble alcohols, for example such as ethanol.

The polymers in dispersion that may be used in the first composition of the invention preferably have a molecular weight of about from 2 000 to 10 000 000 and a Tg of from −100° C. to 300° C., better still from −50° C. to 100° C. and preferably from −10° C. to 50° C.

When the polymer has a glass transition temperature that is too high for the desired use, a plasticizer may be combined therewith so as to lower this temperature of the mixture used. The plasticizer may be chosen from the plasticizers usually used in the field of application, and especially from compounds capable of being solvents for the polymer. Coalescers may also be used so as to help the polymer to form a continuous and uniform deposit. The coalescers or plasticizers that may be used in the invention are especially those mentioned in document FR-A-2 782 917.

It is possible to use film-forming polymers, preferably having a low Tg, of less than or equal to the temperature of the skin and especially less than or equal to 40° C.

Preferably, the polymer used is film-forming, that is to say that it is capable, by itself or in combination with a plasticizer, of forming an isolable film. However, it is possible to use a non-film-forming polymer.

The expression “non-film-forming polymer” means a polymer not capable by itself of forming an isolable film. This polymer makes it possible, in combination with a nonvolatile compound of the oil type, to form a continuous and uniform deposit on the skin and/or the lips.

Among the film-forming polymers that may be mentioned are free-radical, acrylic or vinyl homopolymers or copolymers, preferably with a Tg of less than or equal to 40° C. and especially ranging from −10° C. to 30° C., used alone or as a mixture.

Among the non-film-forming polymers that may be mentioned are free-radical, vinyl or acrylic homopolymers or copolymers, that are optionally crosslinked, preferably with a Tg of greater than 40° C. and especially ranging from 45° C. to 150° C., used alone or as a mixture.

The expression “free-radical polymer” means a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

Monomers bearing an acidic group that may be used include α,β-ethylenic unsaturated carboxylic acids such as acrylic acid, methacrylic acid, crotonic acid, maleic acid and itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), for instance (meth)acrylates of an alkyl, in particular of a C₁-C₂₀ and preferably C₁-C₈ alkyl, (meth)acrylates of an aryl, in particular of a C₆-C₁O aryl, (meth)acrylates of a hydroxyalkyl, in particular of a C₂-C₆ hydroxyalkyl. Alkyl (meth)acrylates that may be mentioned include methyl, ethyl, butyl, isobutyl, 2-ethylhexyl and lauryl (meth)acrylate. Hydroxyalkyl (meth)acrylates that may be mentioned include hydroxyethyl (meth)acrylate and 2-hydroxypropyl (meth)acrylate. Aryl (meth)acrylates that may be mentioned include benzyl or phenyl acrylate.

The (meth)acrylic acid esters that are particularly preferred are the alkyl (meth)acrylates.

Free-radical polymers that are preferably used include copolymers of (meth)acrylic acid and of an alkyl (meth)acrylate, especially of a C₁-C₄ alkyl. More preferably, methyl acrylates optionally copolymerized with acrylic acid may be used.

Amides of acidic monomers that may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide; N-di(C₁-C₄)alkyl-(meth)acrylamides.

The vinyl polymers may also result from the polymerization of ethylenically unsaturated monomers containing at least one amine group, in free form or in partially or totally neutralized form, or alternatively in partially or totally quaternized form. Such monomers may be, for example, dimethylaminoethyl (meth)acrylate, dimethylaminoethylmethacrylamide, vinylamine, vinylpyridine or diallyldimethylammonium chloride.

The vinyl polymers may also result from the homopolymerization or copolymerization of at least one monomer chosen from vinyl esters and styrene monomers. In particular, these monomers may be polymerized with acidic monomers and/or esters thereof and/or amides thereof, such as those mentioned above. Examples of vinyl esters that may be mentioned include vinyl acetate, vinyl propionate, vinyl neodecanoate, vinyl pivalate, vinyl benzoate and vinyl t-butylbenzoate. Styrene monomers that may be mentioned include styrene and α-methylstyrene.

The list of monomers given is not limiting and it is possible to use any monomer known to those skilled in the art falling within the categories of acrylic and vinyl monomers (including monomers modified with a silicone chain).

As other vinyl monomers that may be used, mention may also be made of:

N-vinylpyrrolidone, vinylcaprolactam, vinyl-N—(C₁-C₆) alkylpyrroles, vinyloxazoles, vinylthiazoles, vinylpyrimidines and vinylimidazoles,

olefins such as ethylene, propylene, butylene, isoprene and butadiene.

The vinyl polymer may be crosslinked with one or more difunctional monomers especially comprising at least two ethylenic unsaturations, such as ethylene glycol dimethacrylate or diallyl phthalate.

In a nonlimiting manner, the polymers in dispersion of the invention may be chosen from the following polymers or copolymers: polyurethanes, polyurethane-acrylics, polyureas, polyurea-polyurethanes, polyester-polyurethanes, polyether-polyurethanes, polyesters, polyesteramides, alkyd fatty-chain polyesters; acrylic and/or vinyl polymers or copolymers; acrylic-silicone copolymers; polyacrylamides; silicone polymers, for instance silicone acrylics or polyurethanes, fluoro polymers, and mixtures thereof.

The polymer(s) in dispersion in the liquid phase may represent, as solids, from 2% to 40% of the weight of the composition, preferably from 5% to 30% and better still from 8% to 20%. When the polymer particles in dispersion are surface-stabilized with a stabilizer that is solid at room temperature, the amount of solids in the dispersion represents the total amount of polymer +stabilizer, given that the amount of polymer cannot be less than 2%.

Liquid Phase of the First Composition

According to the invention, the expression “liquid phase” means any aqueous or organic phase that is liquid at room temperature (25° C.) and atmospheric pressure (760 mmHg).

The expression “aqueous phase” means a medium containing water and optionally water-miscible solvents.

The liquid phase is preferably a liquid organic phase.

The expression “liquid organic phase” means any nonaqueous medium that is liquid at room temperature (25° C.) and atmospheric pressure (760 mm Hg), composed of one or more fatty substances that are liquid at room temperature, also known as oils. This organic phase is macroscopically homogeneous (that is to say homogeneous to the naked eye).

This organic phase may contain a volatile liquid organic phase and/or a nonvolatile organic phase.

The expression “nonvolatile organic phase” means any medium capable of remaining on the skin or the lips for several hours. A nonvolatile liquid organic phase in particular has a nonzero vapor pressure at room temperature and atmospheric pressure, of less than 0.02 mm Hg (2.66 Pa) and better still less than 10⁻³ mm Hg (0.13 Pa).

The expression “volatile organic phase” means any nonaqueous medium capable of evaporating from the skin or the lips in less than one hour at room temperature and atmospheric pressure. This volatile phase especially comprises oils with a vapor pressure, at room temperature (25° C.) and atmospheric pressure (760 mm Hg) ranging from 0.02 to 300 mm Hg (2.66 Pa to 40 000 Pa) and preferably from 0.05 to 300 mm Hg (6.65 Pa to 40 000 Pa).

Advantageously, the volatile organic phase contains one or more volatile oils with a flashpoint ranging from 30° C. to 102° C.

The liquid fatty substances or oils of which the organic liquid phase is composed are chosen from oils of mineral, animal, plant or synthetic origin, carbon-based oils, hydrocarbon-based oils, fluoro oils and/or silicone oils, alone or as a mixture provided that they form a macroscopically stable and homogeneous mixture and provided that they are suitable for the intended use.

The expression “hydrocarbon-based oil” means oils predominantly containing carbon atoms and hydrogen atoms and in particular alkyl or alkenyl chains, for instance alkanes or alkenes, but also oils with an alkyl or alkenyl chain comprising one or more alcohol, ether, ester or carboxylic acid groups.

The total liquid organic phase of the first composition can represent from 5% to 98% of the total weight of the composition and preferably from 20% to 85%. Advantageously, it represents at least 30% of the total weight of the composition.

As volatile oils that may be used in the invention, mention may thus be made of hydrocarbon-based oils of mineral or synthetic origin such as linear or branched hydrocarbons, for instance liquid paraffin and its derivatives, liquid petroleum jelly, polydecenes, hydrogenated polyisobutene such as Parleam sold by the company Nippon Oil Fats, squalane of synthetic or plant origin; oils of animal origin, such as mink oil, turtle oil or perhydrosqualene; hydrocarbon-based oils of plant origin with a high triglyceride content consisting of fatty acid esters of glycerol, the fatty acids of which may have varied chain lengths, said chains possibly being linear or branched, and saturated or unsaturated, for instance sweet almond oil, beauty-leaf oil, palm oil, grapeseed oil, sesame oil, arara oil, rapeseed oil, sunflower oil, cottonseed oil, apricot oil, castor oil, alfalfa oil, marrow oil, blackcurrant oil, macadamia oil, musk rose oil, hazelnut oil, avocado oil, jojoba oil, olive oil or cereal germ oil (from corn, wheat, barley or rye); fatty acid esters and especially esters of lanolic acid, of oleic acid, of lauric acid or of stearic acid; synthetic esters of formula R₁COOR₂ in which R₁ represents the linear or branched higher fatty acid residue containing from 7 to 40 carbon atoms and R₂ represents a branched hydrocarbon-based chain containing from 3 to 40 carbon atoms, such as, for example, purcellin oil (cetostearyl octanoate), isononyl isononanoate, C₁₂ to C₁₅ alkyl benzoate, 2-ethylhexyl palmitate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, or glyceryl or diglyceryl triisostearate; hydroxylated esters, for instance isostearyl lactate; pentaerythritol esters; C₈-C₂₆ higher fatty acids such as oleic acid, linoleic acid, linolenic acid or isostearic acid; C₈-C₂₆ higher fatty alcohols such as oleyl alcohol, linoleyl alcohol, linolenyl alcohol, isostearyl alcohol or octyldodecanol; synthetic ethers containing at least 7 carbon atoms, silicone oils such as polydimethylsiloxanes (PDMS) that are liquid at room temperature, linear, and optionally phenylated, such as phenyltrimethicones, phenyltrimethylsiloxydiphenylsiloxanes, diphenyldimethicones, diphenylmethyldiphenyltrisiloxanes, liquid 2-phenylethyl trimethylsiloxysilicates, optionally substituted with aliphatic and/or aromatic groups, for instance alkyl, alkoxy or phenyl groups that are pendent and/or at the end of a silicone chain, these groups containing from 2 to 24 carbon atoms and being optionally fluorinated, or with functional groups such as hydroxyl, thiol and/or amine groups; polysiloxanes modified with fatty acids, with fatty alcohols or with polyoxyalkylenes, for instance dimethicone copolyols or alkylmethicone copolyols; liquid fluorosilicones; or caprylic/capric acid triglycerides, for instance those sold by the company Stearineries Dubois or those sold under the names Miglyol 810, 812 and 818 by the company Dynamit Nobel; and mixtures thereof.

Advantageously, the liquid organic phase may contain one or more organic oils that are volatile at room temperature, for instance volatile cosmetic oils. These oils are favorable toward the production of a deposit with good staying power that is transfer-resistant. After evaporating off these oils, a flexible film-forming deposit that is not sticky on the skin or the lips is obtained. These volatile oils also make it easier to apply the composition to the skin, the lips and integuments. They may be hydrocarbon-based, silicone and/or fluoro oils and may optionally comprise alkyl or alkoxy groups that are pendent or at the end of a silicone chain.

As volatile oils that may be used in the invention, mention may be made of linear or cyclic silicone oils with a viscosity at room temperature of less than 8 mm²/s and especially containing from 2 to 7 silicon atoms, these silicones optionally comprising alkyl or alkoxy groups containing from 1 to 10 carbon atoms. As volatile silicone oils that may be used in the invention, mention may especially be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As other volatile oils that may be used in the invention, mention may be made of hydrocarbon-based volatile oils containing from 8 to 16 carbon atoms and mixtures thereof, and especially C₈-C₁₆ branched alkanes, for instance C₈-C₁₆ isoalkanes (also known as isoparaffins), isododecane, isodecane, isohexadecane and, for example, the oils sold under the trade names “Isopars” or “Permethyls”, and C₈-C₁₆ branched esters, for instance isohexyl neopentanoate, and mixtures thereof.

Advantageously, the volatile organic oil(s) represent(s) from 20% to 90%, preferably from 30% to 80% and better still from 40% to 70% of the total weight of the first composition.

When the liquid organic phase of the first composition contains a volatile oil, this first composition preferably forms the basecoat of the two-coat make-up.

The dispersion of polymer in liquid organic phase may be manufactured as described in document EP-A-0 749 747. The polymerization may be carried out in dispersion, that is to say by precipitation of the polymer during formation, with protection of the particles formed with a stabilizer. In this case, a mixture comprising the initial monomers and also a free-radical initiator is prepared, and this mixture is then dissolved in a medium that is referred to in the rest of the present description as the “organic synthesis medium”. When the liquid phase is organic and contains a nonvolatile oil, the polymerization may be carried out in an apolar organic medium (synthesis medium), followed by addition of the nonvolatile oil (which must be miscible with said synthesis medium) and selective distillation of the synthesis medium.

A synthesis medium is thus chosen such that the initial monomers, and the free-radical initiator, are soluble therein, and the polymer particles obtained are insoluble therein, so that they precipitate therein during their formation. In particular, the synthesis medium can be chosen from alkanes such as heptane, isododecane or cyclohexane.

When the liquid phase of the first composition is a volatile oil, the polymerization may be carried out directly in said oil, which thus also acts as the synthesis medium. The monomers must also be soluble therein, as must the free-radical initiator, and the polymer obtained must be insoluble therein.

The monomers are preferably present in the synthesis medium, before polymerization, in a proportion of 5-20% by weight of the reaction mixture. The total amount of monomers may be present in the medium before the start of the reaction, or a portion of the monomers may be added gradually as the polymerization reaction proceeds.

The free-radical initiator may especially be azobisisobutyronitrile or tert-butylperoxy-2-ethyl hexanoate.

Stabilizer

Advantageously, the polymerization of the polymer in the organic synthesis medium is carried out in the presence of a stabilizer of polymer type.

The polymer particles in organic medium are surface-stabilized, gradually as the polymerization proceeds by means of a stabilizer which may be a block polymer, a grafted polymer and/or random polymer, alone or as a mixture. The stabilization may be carried out by any known means, and in particular by directly adding the block polymer, grafted polymer and/or random polymer during the polymerization.

The stabilizer is preferably also present in the mixture before polymerization. However, it is also possible to add it continuously, especially when the monomers are also added continuously.

2-30% by weight of stabilizer may be used relative to the initial monomer mixture, and preferably 5-20% by weight.

When a grafted and/or block polymer is used as stabilizer, the synthesis solvent is chosen such that at least some of the grafts or blocks of said polymer-stabilizer are soluble in said solvent, the other part of the grafts or blocks not being soluble therein. The polymer-stabilizer used during the polymerization must be soluble, or dispersible, in the synthesis solvent. Furthermore, a stabilizer whose insoluble grafts or blocks have a certain affinity for the polymer formed during the polymerization is preferably chosen.

Among the grafted polymers that may be mentioned are silicone polymers grafted with a hydrocarbon-based chain; hydrocarbon-based polymers grafted with a silicone chain.

Grafted copolymers having, for example, an insoluble backbone of polyacrylic type with soluble grafts of poly(12-hydroxystearic acid) type are also suitable.

It is thus possible to use grafted-block or block copolymers comprising at least one block of polyorganosiloxane type and at least one block of a free-radical monomer, such as grafted copolymers of acrylic/silicone type which may be used especially when the synthesis medium and then the organic liquid phase of the first composition contains a silicone phase.

It is also possible to use grafted-block or block copolymers comprising at least one block of polyorganosiloxane type and at least one block of a polyether. The polyorganopolysiloxane block may especially be a polydimethylsiloxane or a poly(C₂-C₁₈)alkylmethylsiloxane; the polyether block may be a poly(C₂-C₁₈ alkylene), in particular polyoxyethylene and/or polyoxypropylene. In particular, dimethicone copolyols or (C₂-C₁₈)alkyldimethicone copolyols may be used, such as those sold under the name “Dow Corning 3225C” by the company Dow Corning, and lauryl methicones such as those sold under the name “Dow Corning Q2-5200” by the company Dow Corning.

Grafted-block or block copolymers which can also be used are copolymers comprising at least one block resulting from the polymerization of at least one ethylenic monomer, containing one or more optionally conjugated ethylenic bonds, such as ethylene, or dienes such as butadiene or isoprene, and of at least one block of a vinyl, or preferably styrene, polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of ethylene-propylene blocks, and the polymerization of butadiene leads, after hydrogenation, to the formation of ethylene-butylene blocks. Among these polymers which may be mentioned are block copolymers in particular of “diblock” or “triblock” type such as polystyrene/polyisoprene (SI) or polystyrene/polybutadiene (SB), such as those sold under the name ‘Luvitol HSB’ by BASF, of the polystyrene/copoly(ethylene-propylene) (SEP) type, such as those sold under the name ‘Kraton’ by Shell Chemical Co. or alternatively of the polystyrene/copoly(ethylene-butylene) (SEB) type. In particular, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) or Kraton D-1107 (SIS) may be used. Polymers are generally known as hydrogenated or non-hydrogenated diene copolymers.

Gelled Permethyl 99A-750, 99A-753-59 and 99A-753-58 (mixture of triblock and starburst polymer), Versagel 5960 from Penreco (triblock +starburst polymer); OS129880, OS129881 and OS84383 from Lubrizol (styrene/methacrylate copolymer) may also be used.

As grafted-block or block copolymers comprising at least one block resulting from the polymerization of at least one ethylenic monomer with one or more ethylenic bonds, and of at least one block of an acrylic polymer, mention may be made of poly(methyl methacrylate)/polyisobutylene diblock or triblock copolymers or grafted copolymers with a poly(methyl methacrylate) backbone and with polyisobutylene grafts.

As grafted-block or block copolymers comprising at least one block resulting from the polymerization of at least one ethylenic monomer with one or more ethylenic bonds and of at least one block of a polyether such as a C₂-C₁₈ polyalkylene, in particular polyethylenated and/or polyoxypropylenated, mention may be made of polyoxyethylene/polybutadiene or polyoxyethylene/polyisobutylene diblock or triblock copolymers.

When a random polymer is used as stabilizer, it is chosen such that it has a sufficient amount of groups that make it soluble in the intended organic synthesis medium.

Copolymers based on acrylates or methacrylates of alkyls derived from C₁-C₄ alcohols, and acrylates or methacrylates of alkyls derived from C₈-C₃₀ alcohols may thus be used. Mention may be made in particular of the stearyl methacrylate/methyl methacrylate copolymer.

When the synthesis medium is apolar, the stabilizer preferably chosen is a polymer which covers the particles as completely as possible, several stabilizing-polymer chains then becoming adsorbed on a polymer particle obtained by polymerization.

In this case, the stabilizer preferably used is either a grafted polymer or a block polymer, so as to have better interfacial activity. The reason for this is that the blocks or grafts that are insoluble in the synthesis solvent provide more voluminous coverage at the surface of the particles.

When the liquid synthesis medium comprises at least one silicone oil, the stabilizer is preferably chosen from the group consisting of grafted-block or block copolymers comprising at least one block of polyorganosiloxane type and at least one block of a free-radical polymer or of a polyether or a polyester, such as polyoxypropylenated and/or polyoxyethylenated blocks.

When the organic liquid phase does not comprise a silicone oil, the stabilizer is preferably chosen from the group consisting of:

(a) grafted-block or block copolymers comprising at least one block of polyorganosiloxane type and at least one block of a free-radical polymer or of a polyether or a polyester,

(b) copolymers of acrylates or methacrylates of alkyls derived from C₁-C₄ alcohols and of acrylates or methacrylates of alkyls derived from C₈-C₃₀ alcohols,

(c) grafted-block or block copolymers comprising at least one block resulting from the polymerization of at least one ethylenic monomer containing conjugated ethylenic bonds,

and at least one block of a vinyl or acrylic polymer or of a polyether or a polyester, or mixtures thereof.

Diblock polymers are preferably used as stabilizer.

Rheological Agent

Advantageously, the first composition contains one or more rheological agents for structuring and/or gelling its physiologically acceptable medium.

This or these rheological agent(s) is(are) agents capable of thickening and/or gelling the composition. They may be present in an amount that is effective to increase the viscosity of the composition until a solid gel is obtained, that is to say a product that does not run under its own weight, or even a stick.

The rheological agent especially represents from 0.1% to 50% of the total weight of the first composition and better still from 1% to 25%.

This rheological agent is advantageously chosen from lipophilic gelling agents, waxes and fillers, and mixtures thereof.

Lipophilic Gelling Agent

According to one preferred embodiment of the invention, the first composition contains a liquid organic phase; it may thus comprise, as rheological agent, an agent for gelling this liquid organic phase.

Organic-phase gelling agents that may be mentioned include optionally modified clays, for instance hectorites modified with an ammonium chloride of a C₁₀ to C₂₂ fatty acid, for instance hectorite modified with distearyldimethylammonium chloride; fumed silica optionally hydrophobically surface-treated, with a particle size of less than 1 μm; partially or totally crosslinked elastomeric polyorganosiloxanes, of three-dimensional structure, such as those sold under the names KSG6, KSG16, and KSG18 from Shin-Etsu, Trefil E-505C or Trefil E-506C from Dow-Corning, Gransil SR-CYC, SR DMF10, SR-DC556, SR 5CYC gel, SR DMF 10 gel and SR DC 556 gel from Grant Industries, SF 1204 and JK 113 from General Electric; galactomannans comprising from one to six and better from two to four hydroxyl groups per saccharide, substituted with a saturated or unsaturated alkyl chain, for instance guar gum alkylated with C₁ to C₆ and better still C₁ to C₃ alkyl chains, and more particularly ethylated guar having a degree of substitution of 2 to 3, such as the product sold by the company Aqualon under the name N-Hance-AG; ethylcellulose, for instance the products sold under the name Ethocel by Dow Chemical; gums, especially silicone gums, for instance PDMS having a viscosity >100 000 centistokes.

The rheological agent may also be chosen from ethylene homopolymers or copolymers with a weight-average molecular mass of between 300 and 500 000 and better still between 500 and 100 000.

Preferably, the rheological agent is chosen from olefin copolymers of controlled crystallization, as described in patent application EP-A-1 034 776 from the Applicant, such as, for example, the ethylene/octene copolymer sold under the reference Engage 8400 by the company Dupont de Nemours. Specifically, this type of gelling agent gives a film of first composition, and consequently a final make-up result that shows particularly advantageous staying power and transfer-resistance properties.

This or these rheological agent(s) is(are) used, for example, at concentrations of from 0.5% to 20% and better still from 1% to 10% of the total weight of the first composition.

Wax

The rheological agent may also comprise a wax chosen from waxes that are solid at room temperature, such as hydrocarbon-based waxes, for instance optionally modified beeswax, carnauba wax, candelilla wax, ouricurry wax, Japan wax, cork fiber wax or sugar cane wax, paraffin wax, lignite wax, microcrystalline wax, lanolin wax, montan wax, ozokerites, polyethylene wax or ethylene copolymer wax, the waxes obtained by Fischer-Tropsch synthesis, hydrogenated oils, fatty esters and glycerides that are solid at 250C. Silicone waxes may also be used, among which mention may be made of alkyl, alkoxy and/or esters of polymethylsiloxane. The waxes may be in the form of stable dispersions of colloidal wax particles as may be prepared according to known methods, such as those of “Microemulsions Theory and Practice”, L. M. Prince Ed., Academic Press (1977), pages 21-32. Preferably, the waxes used have a melting point at least equal to 45° C.

The waxes may be present in a proportion of from 0.1% to 50% by weight in the first composition and better still from 3% to 25%, so as not to excessively reduce the gloss of this composition and of the film deposited on the lips and/or the skin.

Filler

The rheological agent may furthermore comprise a filler. The term “filler” means any colorless or white particle chosen from mineral or organic, lamellar, spherical or oblong fillers, that are chemically inert in the first composition. Mention may be made of talc, mica, silica, kaolin, polyamide powders, for instance Nylon® powder (Orgasol® from Atochem), poly-β-alanine powders and polyethylene powders, powders of tetrafluoroethylene polymers (Teflon®), lauroyllysine, starch, boron nitride, hollow polymer microspheres such as those of polyvinylidene chloride/acrylonitrile, such as Expancel® (Nobel Industrie), acrylic polymer particles, especially of acrylic acid copolymer, for instance Polytrap® (Dow Corning) and silicone resin microbeads (for example Tospearls® from Toshiba), precipitated calcium carbonate, dicalcium phosphate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate or lithium stearate, zinc laurate and magnesium myristate, and mixtures thereof. These fillers may or may not be surface-treated, especially to make them lipophilic.

Preferably, the fillers have a particle size of less than 50 μm and represent from 0.1% to 35%, preferably from 0.5% to 25% and better still from 1% to 15% of the total weight of the first composition, if they are present.

Second Composition

The cosmetic make-up product according to the invention contains a second composition comprising a second physiologically acceptable medium and a second coloring agent.

According to one preferred embodiment of the invention, the physiologically acceptable medium for the second composition comprises a liquid phase that is nonvolatile at room temperature and atmospheric pressure.

The expression “nonvolatile liquid phase” means any medium capable of remaining on the skin or the lips for several hours. A nonvolatile liquid phase in particular has a vapor pressure at room temperature and atmospheric pressure that is not zero, of less than 0.02 mm Hg (2.66 Pa) and better still less than 10⁻³ mm Hg (2.66 Pa).

The nonvolatile liquid phase of the second composition may contain a hydrocarbon-based phase that is liquid, a fluoro phase that is liquid and/or a silicone phase that is liquid at room temperature.

Preferably, the nonvolatile liquid phase of the second composition in hydrocarbon-based form is characterized by the solubility parameters δD and δa according to the Hansen solubility space satisfying the following conditions: 8≦δD≦22 (J/cm³)^{½}, preferably 12≦δD≦19 (J/cm³)^{1/2}, and better still 16≦δD≦19 (J/cm³)^{½} and 7≦δa≦35 (J/cm³)^{½}, preferably 8<8a<20 (J/cm³)^{1/2}, and better still 8.5≦δa≦12 (J/cm³)^{½}

The definition and calculation of the solubility parameters of the Hansen three-dimensional solubility space are described in the article by C. M. Hansen: “The three dimensional solubility parameters” J. Paint Technol. 39, 105 (1967);

δD characterizes the London dispersion forces derived from the formation of dipoles induced during molecular impacts, and δa=(δH²+δP²)1/2 with

δH which characterizes the specific forces of interaction (hydrogen bonding, acid/base, donor/acceptor, etc. type);

δP which characterizes the Debye interaction forces between permanent dipoles and also the Keesom interaction forces between induced dipoles and permanent dipoles.

The parameters δD and δa are expressed in (J/cm³)^{½}.

The nonvolatile liquid phase may be a mixture of different compounds. In this case, the solubility parameters of the mixture are determined from those of the compounds taken separately, according to the following relationships:

$${δ}_{D mixture} = {\sum }_{i} xi {δ}_{Di} ; {δ}_{ρ mixture} = {\sum }_{i} xi {δ}_{pi} and {δ}_{hmixture} = {\sum }_{i} xi {δ}_{hi}$$

where xi represents the volume fraction of the compound i in the mixture.

It is within the capability of a person skilled in the art to determine the amounts of each compound to obtain a fatty substance mixture that satisfies the above relationships.

As hydrocarbon-based compounds that satisfy these solubility parameters, mention may especially be made of the following compounds:

| | δD | δa |
|---|---|---|
| diisostearyl malate | 16.61 | 7.19 |
| octyldodecanol | 16.36 | 7.70 |
| propylene glycol monoisostearate | 16.36 | 8.74 |
| polyglyceryl-2 diisostearate | 16.79 | 9.07 |
| castor oil | 16.79 | 9.09 |
| polyglyceryl-3 diisostearate | 16.96 | 10.40 |
| polyglyceryl-2 isostearate | 17.03 | 13.25 |
| butylene glycol | 16.65 | 22.83 |
| propylene glycol | 15.95 | 25.02 |
| glycerol | 17.81 | 31.73 |
| and mixtures thereof | | |

When the nonvolatile liquid phase of the second composition contains a fluoro phase, it comprises at least one fluoro compound chosen from fluorosilicone compounds, fluoro polyethers and/or fluoroalkanes.

Preferably, the nonvolatile liquid phase of the second composition comprises at least one fluorosilicone compound of formula (I):

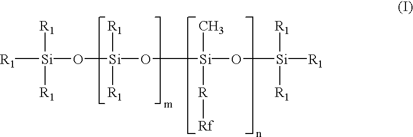

in which:

R represents a linear or branched divalent alkyl group containing 1 to 6 carbon atoms, preferably a divalent methyl, ethyl, propyl or butyl group,

Rf represents a fluoroalkyl radical, especially a perfluoroalkyl radical, containing 1 to 9 carbon atoms, preferably 1 to 4 carbon atoms,

R₁ represent, independently of each other, a C₁-C₂₀ alkyl radical, a hydroxyl radical or a phenyl radical,

m is chosen from 0 to 150 and preferably from 20 to 100, and

n is chosen from 1 to 300 and preferably from 1 to 100.

Fluorosilicone compounds of formula (I) that may especially be mentioned are those sold by the company Shin Etsu under the names “X22-819”, “X22-820”, “X22-821” and “X22-822” or “FL-100”.

As another fluoro compound that may form part of the composition of the fluoro phase of the second composition, mention may especially be made of the fluoro polyethers of formula (II) below:

R₆—(CF₂—CFR₃—CF₂—O)p—(CFR₄—CF₂—O)q—(CFR₅—O)r—R₇ (II)

in which:

—R₃ to R₆ represent, independently of each other, a monovalent radical chosen from —F, —(CF₂)n—CF₃ and —O—(CF₂)n—CF₃,

R₇ represents a monovalent radical chosen from —F and —(CF₂)n—CF₃,

with n ranging from 0 to 4 inclusive,

p ranging from 0 to 600, q ranging from 0 to 860, r ranging from 0 to 1 500, and p, q and r being integers chosen such that the weight-average molecular mass of the compound ranges from 500 to 100 000 and preferably from 500 to 10 000.

Such compounds are especially described in document EP-A-0 196 904.

Among the commercial products that may be used in the present invention as fluoro compound, mention may be made of the Fomblins from the company Montefluos, and the Demnum S products from the company Daikin Industries.

As fluoro compounds that may be used in the context of the present invention, mention may also be made of fluoroalkanes, such as C₂-C₅₀ and especially C₅-C₃₀ perfluoroalkanes and fluoroalkanes, such as perfluorodecalin, perfluoroadamantane and bromoperfluorooctyl, and mixtures thereof.

When the nonvolatile liquid phase of the second composition contains a silicone phase, it comprises advantageously at least one silicone oil and preferably a phenylsilicone oil.

The phenylsilicone oils that may be used according to the present invention have a viscosity measured at 25° C. and atmospheric pressure ranging from 5 to 100 000 cSt and preferably from 5 to 10 000 cSt.

The silicone oil may be, for example, a phenyl trimethicone, a phenyl dimethicone, a phenyl trimethylsiloxydiphenylsiloxane, a diphenyl dimethicone, a diphenylmethyldiphenyltrisiloxane or a mixture of different phenylsilicone oils, and in particular may correspond to formula (A) below:

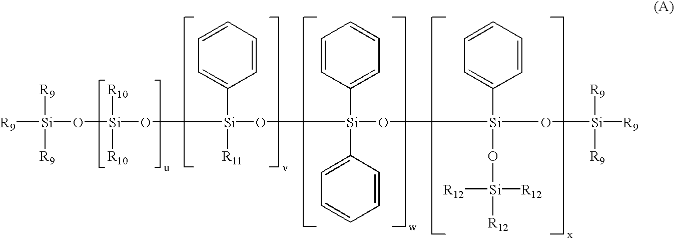

in which:

R₉ and R₁₂ are each independently a C₁-C₃₀ alkyl radical, an aryl radical or an aralkyl radical,

R₁₀ and R₁₁, are each independently a C₁-C₃₀ alkyl radical or an aralkyl radical,

u, v, w and x are each independently integers ranging from 0 to 900, with the proviso that the sum v+w+x is other than 0 and that the sum u+v+w+x ranges from 1 to 900 and in particular u+v+w+x ranges from 1 to 800.

Advantageously, R₉ is a C₁-C₂₀ alkyl radical, a phenyl radical or an aralkyl radical of the type R′-C₆H₅, R′ being a C₁-C₅ alkyl, R₁₀ and R₁₁, are each independently a C₁-C₂₀ alkyl radical or an aralkyl radical of the type R′-C₆H₅, R′ being a C₁-C₅ alkyl, and R₁₂ is a C₁-C₂₀ alkyl radical.

Preferably, R₉ is a methyl, ethyl, propyl, isopropyl, decyl, dodecyl or octadecyl radical, or a phenyl, tolyl, benzyl or phenethyl radical, R₁₀ and R₁₁ are each independently a methyl, ethyl, propyl, isopropyl, decyl, dodecyl or octadecyl radical, or a tolyl, benzyl or phenethyl radical, and R₁₂ is a methyl, ethyl, propyl, isopropyl, decyl, dodecyl or octadecyl radical.

According to one preferred embodiment of the invention, the non-volatile liquid phase of the second composition contains a silicone-based phase comprising a phenylsilicone oil with a viscosity of less than 500 cSt at 25° C., referred to as a “low-viscosity phenylsilicone oil”, and a phenylsilicone oil with a viscosity at least equal to 500 cSt at 25° C., referred to as a “high-viscosity phenylsilicone oil”. Advantageously, the low-viscosity phenylsilicone oil has a viscosity at 25° C. ranging, for example, from 5 to 499 cSt, preferably from 5 to 300 cSt and better still from 5 to 100 cSt, and the high-viscosity phenylsilicone oil has a viscosity at 25° C. ranging, for example, from 500 to 10 000 cSt, preferably from 600 to 5 000 cSt and better still from 600 to 3 000 cSt.

The use of low-viscosity and high-viscosity phenylsilicone oils as defined above makes it possible to obtain, after application to the skin, the lips and/or integuments, a film of composition that is particularly glossy, uniform and of good staying power.

Preferably, these low-viscosity and high-viscosity phenylsilicone oils satisfy formula (A). Preferably, the first low-viscosity phenylsilicone oil satisfies formula (A) with the sum u+v+w+x ranging from 1 to 150, better still from 1 to 100 or even from 1 to 50, and the second high-viscosity phenylsilicone oil satisfies formula (A) with the sum u+v+w+x ranging from 151 to 900, better still from 160 to 800, or even from 160 to 500.

In particular, the low-viscosity phenylsilicone oil satisfies formula (III) below:

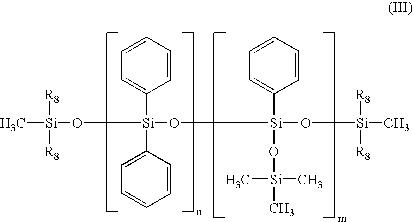

in which

R₈ is a C₁-C₃₀ alkyl radical, an aryl radical or an aralkyl radical,

n is an integer ranging from 0 to 100, and better still, of less than 100,

m is an integer ranging from 0 to 100, with the proviso that the sum m+n ranges from 1 to 100, and better still, is less than 100.

Advantageously, R₈ is a C₁-C₂₀ alkyl radical, a phenyl radical or an aralkyl radical of the type R′-C₆H₅, R′ being a C₁-C₅ alkyl.

Preferably, R₈ is a methyl, ethyl, propyl, isopropyl, decyl, dodecyl or octadecyl radical, or alternatively a phenyl, tolyl, benzyl or phenethyl radical. R₈ is advantageously a methyl radical.

Among the low-viscosity phenylsilicone oils that may be used in the invention, mention may be made of the oils DC556 (22.5 cSt), or SF558 (10-20 cSt) from Dow Corning, the oil Abil AV8853 (4-6 cSt) from Goldschmidt, the oil Silbione 70 633 V 30 (28 cSt) from Rhône-Poulenc, the oils 15 M 40 (50 to 100 cSt) or 15 M 50 (20 to 25 cSt) from PCR, the oils SF 1550 (25 cSt) or PK 20 (20 cSt) from Bayer, the oil Belsil PDM 200 (200 cSt) from Wacker, the oils KF/53 (175 cSt), KF 54 (400 cSt) and KF 56 (14 cSt) from Shin-Etsu. Among the high-viscosity phenylsilicone oils that may be used in the invention, mention may be made of the oils 15 M 30 from PCR (500 cSt) or Belsil PDM 1000 (1000 cSt) from Wacker.

The values in parentheses represent the viscosities at 25° C.

The weight ratio between the low-viscosity phenylsilicone oil and the high-viscosity phenylsilicone oil can range, for example, from 70/30 to 30/70, better still from 60/40 to 40/60 and even better still from 55/45 to 45/55.

Preferably, the nonvolatile liquid phase of the second composition contains a silicone phase that is liquid at room temperature.

The nonvolatile liquid phase of the second composition represents from 1% to 100%, preferably from 5% to 95%, better still from 20% to 80% and even better still from 40% to 80% of the total weight of the second composition.

The physiologically acceptable medium of the second composition may also contain a volatile liquid phase whose rate of evaporation is different than the rate of evaporation of the volatile phase of the first composition, and in particular the rate of evaporation of the volatile phase of the second composition is less than the rate of evaporation of the volatile phase of the first composition.

Coloring Agents

The first (or the second) composition of the cosmetic make-up product according to the invention contains one or more goniochromatic coloring agents chosen from mesomorphic coloring agents or liquid-crystal coloring agents (referred to as LC agents) and multilayer interference structures. Preferably, only one goniochromatic agent is used for ease of implementation and reduced manufacturing costs.

The LC agents are especially linear or cyclic monomers or polymers onto which are grafted mesomorphic groups, especially cholesteric or nematic groups. The LC coloring agents comprise, for example, silicones or cellulose ethers onto which are grafted mesomorphic groups.

The LC coloring agents are chosen in particular from cyclic polyorganosiloxanes grafted with cholesteric and biphenyl groups. These grafted polyorganosiloxanes are in particular crosslinked in a three-dimensional structure.

These LC coloring agents are especially chosen from the cyclic silicones obtained by polymerization of the monomer of formula (IV) below:

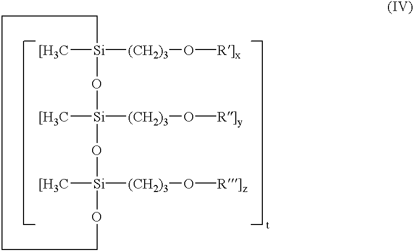

in which:

0≦x≦1 (preferably 1); 0≦y≦1 (preferably 1);

0≦z≦1 (preferably 1) with x+y+z≠0; 3≦t≦10;

R′ denotes a group of the following formula:

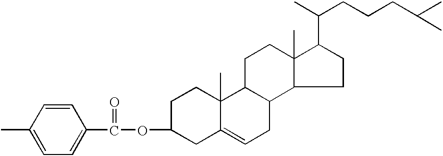

R″ denotes a group of the following formula:

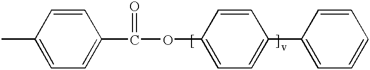

R″′ denotes a group of the following formula:

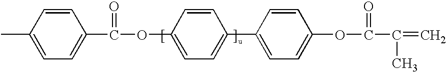

with u and v=0 or 1, independently of each other, with one or more monomers chosen from cholesteryl methacrylate, cholesteryl 4-(2-propen-1-oxy)benzoate, cholesteryl 4-(2-propen-1-oxy)benzoate,

4-methacryloyloxyphenyl 4-(2-propen-1-oxy)benzoate and the monomers described in patents U.S. Pat. No. 5,362,315 (especially Example 2) and U.S. Pat. No. 5,851,604, and mixtures thereof.

These compounds are generally in the form of amorphous white powders. They have the particular feature of displaying the color change effect depending on the direction of observation only as a function of the support (and especially of its color) onto which it is spread.

Examples of LC coloring agents that may be mentioned in particular are the “LC pigments” from the company Wacker known as SLM 41101 (blue/green), SLM 41102 (red/gold) and SLM 41103 (yellow/green) and LC Pigment Green 516 S (blue/green).

The goniochromatic agents of multilayer structure are especially the ones described in the following documents: U.S. Pat. No. 3,438,796, EP-A-227 423, U.S. Pat. No. 5,135,812, EP-A-170 439, EP-A-341 002, U.S. Pat. Nos. 4,930,866, 5,641,719, EP-A-472 371, EP-A 395 410, EP-A-753 545, EP-A-768 343, EP-A-571 836, EP-A-708 154, EP-A-579 091, U.S. Pat. Nos. 5,411,586, 5,364,467, WO-A-97/39066, DE-A-4 225 031, WO 95/17479 (BASF), DE-A-196 14 637. They are in the form of flakes of metalized color.

The multilayer structures that may be used in the invention are, for example, the following structures: Al/SiO₂/Al/SiO₂/Al; Cr/MgF₂/Al/MgF₂/Al; MOS₂/SiO₂/Al/SiO₂/MOS₂; Fe₂O₃/SiO₂/Al/SiO₂/Fe₂O₃; Fe₂O₃/SiO₂/Fe₂O₃/SiO₂/Fe₂O₃; MOS₂/SiO₂/mica-oxide/SiO₂/MOS₂; Fe₂O₃/SiO₂/mica-oxide/SiO₂/Fe₂O₃. Different colors are obtained depending on the thickness of the various coats. Thus, with the Fe₂O₃/SiO₂/Al/SiO₂/Fe₂O₃ structure, the color changes from green-gold to red-gray for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Birefringent multilayer structures comprising an alternation of polymer layers of the polyethylene naphthalate and polyethylene terephthalate type, as described in document WO-A-96/19347, may also be used.

The second (or first) composition of the invention may comprise one or more monochrome coloring agents chosen from liposoluble or water-soluble monochrome coloring agents, monochrome pigments and nacres conventionally used in cosmetic compositions, and combinations thereof.

According to one embodiment of the invention, the monochrome coloring agent may be chosen such that it has one of the colors of the goniochromatic coloring agent or of the set of goniochromatic agents.

The term “pigments” should be understood as meaning white or colored, mineral or organic particles that are insoluble in the liquid fatty phase, intended to color and/or opacify the composition. The term “nacres” should be understood as meaning iridescent particles, especially produced by certain mollusks in their shell or alternatively synthesized. The term “dyes” should be understood as meaning generally organic compounds that are soluble in the fatty substances, for instance the oils, or in an aqueous-alcoholic phase.

As monochrome mineral pigments that may be used in the invention, mention may be made of titanium oxide, zirconium oxide or cerium oxide, and also zinc oxide, iron oxide or chromium oxide and ferric blue. Among the organic pigments that may be used in the invention, mention may be made of carbon black and barium, strontium, calcium and aluminum lakes.

The dyes may be liposoluble or water-soluble. The liposoluble dyes are, for example, Sudan red, DC Red 17, DC Green 6, β-carotene, soybean oil, Sudan brown, DC Yellow 11, DC Violet 2, DC Orange 5, quinoline yellow and annatto. They may represent from 0.01% to 20% of the total weight of each first and/or second composition and better still from 0.1% to 10%. The water-soluble dyes are especially copper sulfate, iron sulfate, water-soluble sulfopolyesters such as those described in documents FR-96 154 152, rhodamines, natural dyes (carotene or beetroot juice), methylene blue and caramel.

The nacres may be present in the first and/or the second composition in a proportion of from 0% to 20% relative to the total weight of each composition, and preferably in a proportion of about from 1% to 15%. Among the nacres that may be used in the first and/or second composition, mention may be made of mica coated with titanium oxide, with iron oxide, with natural pigment or with bismuth oxychloride, such as colored titanium mica.

In general, the first and second coloring agents represent from 0.001% to 60% of the total weight of the first or the second composition, respectively, preferably from 0.01% to 50% and better still from 0.1% to 40%. For pulverulent compositions, the amount of coloring agents may be up to 85% and even up to 98%.

The coloring agent or the filler may also be present in the form of a “particulate paste”.

Particulate Paste

For the purposes of the invention, the expression “particulate paste” means a concentrated dispersion of coated or uncoated particles in a continuous medium, stabilized with a dispersant or optionally without a dispersant. These particles may be chosen from pigments, nacres, solid fillers and mixtures thereof. These particles may be of any shape, especially of spherical or elongated shape, for instance fibers. They are insoluble in the medium.

The dispersant serves to provent the dispersed particles from aggregating or flocculating. The dispersant concentration generally used to stabilize a dispersion is from 0.3 to 5 mg/m² and preferably from 0.5 to 4 mg/m² of surface area of particles. This dispersant may be a surfactant, an oligomer, a polymer or a mixture of several of them, bearing one or more functionalities having a strong affinity for the surface of the particles to be dispersed. In particular, they may attach physically or chemically to the surface of pigments. These dispersants also contain at least one functional group that is compatible with or soluble in the continuous medium. In particular, poly(12-hydroxystearic acid) such as that sold under the reference Arlacel P100 by the company Uniqema, esters of (12-hydroxystearic acid) such as the stearate of poly(12-hydroxystearic acid) with a molecular weight of about 750 g/mol sold under the name Solsperse 21 000 by the company Avecia, esters of poly(12-hydroxystearic acid) with polyols such as glycerol, diglycerine such as the polyglyceryl-2 dipolyhydroxystearate (CTFA name) sold under the reference Dehymuls PGPH by the company Henkel.

As other dispersants which may be used in the composition of the invention, mention may be made of quaternary ammonium derivatives of polycondensed fatty acids, for instance Solsperse 17 000 sold by the company Avecia, and mixtures of polydimethylsiloxane/oxypropylene, such as those sold by the company Dow Corning under the references DC2-5185 and DC2-3225 C.

Poly(12-hydroxystearic acid) and the poly(12-hydroxystearic acid) esters are preferably intended for a hydrocarbon-based or fluorinated medium, whereas the mixtures of oxyethylenated/oxypropylenated dimethylsiloxane are preferably intended for a silicone medium.

The dispersion is a suspension of particles that are generally micron-sized (<10 μm) in a continuous medium. The volume fraction of particles in a concentrated dispersion is from 20% to 40% and preferably greater than 30%, which corresponds to a weight content that may be up to 70% depending on the density of the particles.

The particles dispersed in the medium may consist of mineral or organic particles or mixtures thereof, such as those described below.

The continuous medium of the paste may be of any nature and may contain any solvent or liquid fatty substance and mixtures thereof. Advantageously, the liquid medium of the particulate paste is one of the liquid fatty substances or oils that it is desired to use in the first or second composition, thus forming part of the liquid organic phase of the first or second composition.

Advantageously, the “particulate paste” is a “pigmentary paste” containing a dispersion of coated or uncoated colored particles. These colored particles are pigments, nacres or a mixture of pigments and/or nacres such as those described above.

Preferably, the coloring agent for the first and/or the second composition is in the form of a dispersion or particulate paste as described above.

Advantageously, the dispersion represents from 0.5% to 60% by weight of each first and/or second composition, better still from 2% to 40% and even better still from 2% to 30%.

Additives

The first and second compositions in the make-up product according to the invention may also contain one or more cosmetic, dermatological, hygiene or pharmaceutical active agents.

As cosmetic, dermatological, hygiene or pharmaceutical active agents that may be used in the compositions according to the invention, mention may be made of moisturizers (polyols, for instance glycerol), vitamins (C, A, E, F, B or PP), essential fatty acids, essential oils, ceramides, sphingolipids, liposoluble sunscreens or sunscreens in the form of nanoparticles, and specific skin-treatment active agents (protective agents, antibacterial agents, antiwrinkle agents, etc.).

These active agents are used in an amount that is usual for a person skilled in the art and especially at concentrations of from 0% to 20% and especially from 0.001% to 15% relative to the total weight of the first or second composition.

Each composition in the product according to the invention may furthermore comprise, depending on the intended type of application, the constituents conventionally used in the fields under consideration, which are present in an amount that is suitable for the desired presentation form.

In general, the physiologically acceptable media for each of the first and second compositions in the product according to the invention may comprise, in addition to the liquid phase, the polymer dispersion and the coloring agent for the first composition and the nonvolatile liquid phase for the second composition, additional fatty substances that may be chosen from waxes, oils, gums and/or pasty fatty substances, that are hydrocarbon-based, silicone-based and/or fluoro-based, of plant, animal, mineral or synthetic origin, and mixtures thereof.

Preferably, the physiologically acceptable medium for the first composition contains a gum, preferably a silicone gum with a viscosity at room temperature ranging from 50 000 to 10⁷ cSt and preferably from 100 000 to 10⁶ cSt.

Preferably, the physiologically acceptable medium for the second composition contains a pasty fatty substance and/or a wax chosen from the waxes mentioned above.

Each composition of the product according to the invention may also contain any other additive usually used in such compositions, for instance oil thickeners or aqueous-phase thickeners (acrylic gelling agent), antioxidants, fragrances, preserving agents (pentylene glycol), surfactants or liposoluble polymers (for example polyvinylpyrrolidone/eicosene copolymer).

When the physiologically acceptable medium for the first and/or the second composition contains a liquid organic phase, this medium may especially contain water dispersed or emulsified in said liquid organic phase.

In one specific embodiment of the invention, the compositions of the process according to the invention can be prepared in the usual manner by a person skilled in the art. They can be in the form of a cast product and, for example, in the form of a stick or tube, in the form of a soft paste in a heating bag or in the form of a dish which can be used by direct contact or with a sponge. In particular, they constitute, alone or combined, a cast foundation, a cast in particular colored, face powder or eye shadow, a lipstick, a lip gloss or a concealer product. They can also be in the form of a soft paste or alternatively in the form of a gel or a more or less fluid cream. In this case, they can constitute foundations or lipsticks that are fluid or pasty, lip glosses, antisun products or skin-coloring products, eyeliner or body make-products, or alternatively they may have care properties and may then be in the form of a lipcare balm or base.

Each composition in the product according to the invention may be in any presentation form normally used for topical application and especially in the form of an oily or aqueous solution, an oily or aqueous gel, an oil-in-water or water-in-oil emulsion, a multiple emulsion or a dispersion of oil in water by means of vesicles, the vesicles being located at the oil/water interface, or a powder. Each composition may be fluid or solid.

Advantageously, the first or second composition, or both of them, have a continuous fatty phase and are preferably in anhydrous form and may contain less than 5% water, and better still less than 1% water, relative to the total weight of the first or second composition. In particular, the whole two-coat make-up product is in anhydrous form.

Each composition may have the appearance of a lotion, a cream, an ointment, a soft paste, a salve, a cast or molded solid, which is especially in stick or dish form, or alternatively a compacted solid.

Preferably, each composition is in the form of a more or less rigid stick.

Each composition may be packaged separately in the same packaging article, for example in a two-compartment pen, the base composition being delivered from one end of the pen and the top composition being delivered from the other end of the pen, each end being closed, especially in a leaktight manner, by a cap.

Preferably, the composition that is applied as a first coat is in solid form, thus allowing a more practical application, better stability over time and at elevated temperature for the composition, and allows the make-up to be applied in a precise line, which is highly desirable in the case of a lipstick or an eyeliner.

The product according to the invention may advantageously be used for making up the skin and/or the lips and/or integuments depending on the nature of the ingredients used. In particular, each composition of the invention may be in the form of a tube of lipstick or lipstick paste, a solid foundation, a concealer product or products for the contours of the eyes, an eyeliner, a mascara, an eyeshadow, a body make-up product or a skin coloring product.

Preferably, the product according to the invention is a make-up product for the skin or the lips.

The product is in particular in the form of a lipstick.

Preferably, the second composition is in solid form.

Advantageously, the topcoat has care properties.

The compositions of the invention may be obtained by heating the various constituents to the temperature of the highest-melting waxes, followed by casting the molten mixture in a mold (dish or glove digit). They may also be obtained by extrusion, as described in patent application EP-A-0 667 146.

The examples below are intended to illustrate the present invention in a nonlimiting manner.

### EXAMPLES

The amounts are given as percentages by mass.

### Example 1

### Polymer Dispersion

A dispersion of noncrosslinked copolymer of methyl acrylate and of acrylic acid in a 95/5 ratio in isododecane is prepared, according to the method of Example 1 of document EP-A-749 746, replacing the heptane with isododecane. A dispersion of poly(methyl acrylate/acrylic acid) particles surface-stabilized in isododecane with a polystyrene/copoly(ethylene-propylene) block diblock copolymer sold under the name Kraton G1701 (Shell), having a solids content of 24.6% by weight and a mean particle size of 180 nm (polydispersity: 0.05%) and a Tg of 20° C., is thus obtained. This copolymer is capable of forming a film.

### Example 2

### Pigmentary Paste

The pigmentary paste used is a mixture of 3 pigmentary pastes each containing a different pigment:

| Paste No. 1: | |
|---|---|
| DCRed No. 7 | 30% |
| Poly (12-hydroxystearic acid) stearate | 2% |
| (Solsperse 21000) | |
| Hydrogenated polyisobutene (Parleam) | 68% |
| Paste No. 2: | |
| Yellow No. 6 Al lake | 50% |
| Poly (12-hydroxystearic acid) stearate | 2% |
| (Solsperse 21000) | |
| Hydrogenated polyisobutene (Parleam) | 48% |
| Paste No. 3: | |
| Titanium dioxide | 70% |
| Poly (12-hydroxystearic acid) stearate | 1% |
| (Soisperse 21000) | |
| Hydrogenated polyisobutene (Parleam) | 29% |

A pigmentary paste containing a mixture of 10% of paste No. 1, 2% of paste No. 2 and 2.14% of paste No. 3 is produced.

### Example 3

### Lip Make-Up Product

First composition

| | |
|---|---|
| Phase A | |
| polymer particle dispersion | 71% |
| ethylene/octene copolymer (76/24) sold under | 3.50% |
| the reference Engage 8400 by the company | |
| Dupont de Nemours | |
| Phase B | 4.14% |
| pigmentary paste according to Example 2 | |
| Phase C | 10% |
| polytetrafluoroethylene | |
| Phase D | 1.36% |
| cyclopentasiloxane | |

Procedure

Phase A is obtained by dissolving the gelling agent ethylene/octene copolymer) at 110° C. in the polymer particle dispersion over about one hour using a Raynerie mixer.

After homogenization, the temperature is allowed to return to about 30° C. and phases B, C and D are then successively added with stirring using a Raynerie mixer. The first composition is then packaged in a heating bag at room temperature. It is in the form of a soft paste.

Second composition

| | |
|---|---|
| castor oil | 74% |
| PEG-45 dodecyl glycol copolymer | 10% |
| octacosanyl stearate (wax) | 8% |
| multilayer interference pigment sold under | 8% |
| the reference Sicopearl Fantastico Or by the | |
| company BASF | |

Procedure

The constituents are weighed out together and heated at 100° C. until the wax has completely melted. After homogenization, the composition can be cast in a suitable mold to obtain a stick in the form of a “pen”.

A first coat of the first composition is applied to the lips using a felt, it is left to dry for 3 minutes and a second coat of the second composition is then applied over this first coat.

This two-coat make-up is comfortable, does not cause dehydration and has good staying power, it shows little transfer and does not migrate. These properties were checked and confirmed by qualified individuals.

### Example 4

### Lip Make-Up Product

First composition

| | |
|---|---|
| Phase A | 69.16% |
| polymer particle dispersion of Example 1 | |
| Phase B | 14.14% |
| pigmentary paste of Example 2 | |
| Phase C | |
| polyethylene wax (Mw* = 500) | 12% |
| ozokerite | 3.20% |
| linear fatty alcohol sold under the reference | |
| Performacol 550 by the company New Phase | |
| Technologies | 1.50% |

*Mw represents the weight-average molecular mass.

Procedure

The constituents are all weighed out together and mixed at 100-105° C. using a Raynerie mixer. After homogenization, the composition is cast at 100° C. in a mold and packaged in the form of a “pen”.

Second composition

| | |
|---|---|
| phenyltrimethicone (of viscosity equal to | 42% |
| 20 cSt) sold under the reference DC 556 by | |
| the company Dow Corning | |
| phenyltrimethicone (of viscosity equal to | 42% |
| 1 000 cSt) sold under the reference Belsil | |
| PDM 1000 by the company Wacker | |
| polyethylene wax (Mw = 500) | 8% |
| multilayer interference pigment sold under | 8% |
| the reference Sicopearl Fantastico Or by the | |
| company BASF | |

Procedure

The constituents are weighed out together and heated at 100° C. until the wax has completely melted.

After homogenization, the composition may be cast in a suitable mold to obtain a stick in the form of a “pen”.

A first coat of the first composition is applied to the lips and is left to dry for 3 minutes. A second coat of the second composition is then applied as a topcoat over this first coat.

This two-coat make-up is comfortable and has good staying power, it shows little transfer and does not migrate. These properties were checked and confirmed by qualified individuals.

These products are easy to remove with standard waterproof make-up remover such as Bifacil sold by the company Lancôme.

The present application claims priority to French application 0104939, filed Apr. 10, 2001, which is incorporated herein by reference.

1. A product, comprising
a first composition, which contains polymer particles dispersed in a first physiologically acceptable medium which is surface-stabilized by a stabilizer in a liquid phase, and at least one first coloring agent; and
a second composition which contains, in a second physiologically acceptable medium, at least two second coloring agents, wherein at least one of the coloring agents is a goniochromatic agent that produces different colors under different light and the angle of observation; and, wherein at least one second coloring agent is a monochromic coloring agent.

2. A product, comprising
a first composition, which contains polymer particles dispersed in a first physiologically acceptable medium, which is surface-stabilized by a stabilizer in a liquid organic phase, and at least one first coloring agent; and
a second composition containing, in a second physiologically acceptable medium, at least two second coloring agents, wherein at least one of the coloring agents is a goniochromatic agent that produces different colors under different light and the angle of observation, and wherein at least one of the second coloring agents is a monochromic agent.

3. The product as claimed in claim 1, wherein the polymer particles have a mean size of from 5 to 800 nm.

4. The product as claimed in claim 1, wherein the polymer particles are insoluble in a water-soluble alcohol.

5. The product as claimed in claim 1, wherein the polymer particles are selected from the group consisting of polyurethanes, polyurethane-acrylics, polyureas, polyurea-polyurethanes, polyester-polyurethanes, polyether-polyurethanes, polyesters, polyesteramides, alkyd fatty-chain polyesters, acrylic polymers, acrylic copolymers, vinyl polymers, vinyl copolymers acrylic-silicone copolymers polyacrylamides, silicone polymers, fluoro polymers, and mixtures thereof.

6. The product as claimed in claim 1, wherein the polymer can form a film.

7. The product as claimed in claim 1, wherein the polymer, as a solid, is in an amount from 2% to 40% relative to the total weight of the first composition.

8. The product as claimed in claim 1, wherein the polymer, as a solid, is in an amount from 5% to 30%.

9. The product as claimed in claim 1, wherein the polymer, as a solid, is in an amount from 8% to 20%.

10. The product as claimed in claim 1, wherein the stabilizer is selected from the group consisting of block polymers, grafted polymers, random polymers, and blends thereof.

11. The product as claimed in claim 1, wherein the stabilizer is selected from the group consisting of:
silicone polymers grafted with a hydrocarbon chain;
hydrocarbon polymers grafted with a silicone chain;
grafted copolymers with an insoluble backbone of polyacrylic and soluble grafts of poly(12-hydroxystearic acid);
grafted-block copolymers with at least one polyorganosiloxane block and at least one free-radical polymer block;
block copolymers with at least one polyorganosiloxane block and at least one free-radical polymer block;
grafted copolymers with at least one polyorganosiloxane block and at least one polyether block;
block copolymers with at least one polyorganosiloxane block and at least one polyether block;
copolymers of C₁-C₄ alkyl acrylates;
copolymers of C₁-C₄ methacrylates;
copolymers of C₈-C₃₀ alkyl acrylates;
copolymers of C₈-C₃₀ alkyl methacrylates;
grafted-block copolymer with at least one block of polymerized ethylenic monomers and at least one vinyl polymer;
grafted-block copolymer with conjugated bonds, at least one block of polymerized ethylenic monomers and at least one vinyl polymer;
block copolymers with at least one block of polymerized ethylenic monomers and at least one vinyl polymer;
block copolymers with conjugated bonds, at least one block of polymerized ethylenic monomers and at least one vinyl polymer;
grafted-block copolymer with at least one block of polymerized ethylenic monomers and at least one acrylic polymer;
grafted-block copolymer with conjugated bonds, at least one block of polymerized ethylenic monomers and at least one acrylic polymer;
block copolymers with at least one block of polymerized acrylic monomers and at least one vinyl polymer;
block copolymers with conjugated bonds, at least one block of polymerized ethylenic monomers and at least one acrylic polymer;
grafted-block or block copolymers comprising at least one polymerized diene and at least one polyether block; and
mixtures thereof.

12. The product as claimed in claim 1, wherein the stabilizer is a grafted-block polymer with at least one polymerized diene and at least one vinyl polymer; or a block polymer with at least one polymerized diene and at least one vinyl polymer.

13. The product as claimed in claim 1, wherein the stabilizer is a diblock polymer.

14. The product as claimed in claim 1, wherein the liquid phase of the first composition comprises a liquid organic phase.

15. The product as claimed in claim 2, wherein the liquid organic phase comprises at least one organic oil that is volatile at room temperature and atmospheric pressure.

16. The product as claimed in claim 15, wherein the volatile organic oil is in amount from 20% to 90% of the total weight of the first composition.

17. The product as claimed in claim 15, wherein the volatile organic oil is in an amount from 30% to 80% of the total weight of the first composition.

18. The product as claimed in claim 15, wherein the volatile organic oil is in an amount from 40% to 70% of the total weight of the first composition.

19. The product as claimed in claim 1, wherein the first composition further comprises a rheological agent which is selected from group consisting of lipophilic gelling agents, waxes, fillers, and mixtures thereof, wherein the rheological agent structures or gels the physiologically acceptable medium.

20. The product as claimed in claim 19, wherein the rheological agent is in an amount from 0.1% to 50% of the total weight of the first composition.

21. The product as claimed in claim 19, wherein the rheological agent is in an amount from 1% to 25% of the total weight of the first composition.

22. The product as claimed in claim 19, wherein the lipophilic gelling agent is selected from the group consisting of ethylene homopolymers with a weight average molecular weight of from 300 to 500,000; ethylene copolymers with a weight average molecular weight of from 300 to 500,000; ethylene homopolymers with a weight average molecular weight of from 500 to 100,000; ethylene copolymers with a weight average molecular weight of from 500 to 100,000; and mixtures thereof.

23. The product as claimed in claim 19, wherein the rheological agent is an olefin copolymer of controlled crystallization.

24. The product as claimed in claim 19, wherein the rheological agent is an ethylene-octene copolymer.

25. The product as claimed in claim 19, wherein the rheological agent is in an amount from 0.5% to 20% of the total weight of the first composition.

26. The product as claimed in claim 19, wherein the rheological agent is in an amount from 1% to 10% of the total weight of the first composition.

27. The product as claimed in claim 19, wherein the rheological agent is a wax.

28. The product as claimed in claim 27, wherein the wax is in an amount of from 0.1% to 50% by weight of the first composition.

29. The product as claimed in claim 27, wherein the wax is in an amount of from 3% to 25% of the first composition.

30. The product as claimed in claim 18, wherein the rheological agent further comprises a filler.

31. The product as claimed in claim 30, wherein the filler has a particle size of less than 50 μm.

32. The product as claimed in claim 30, wherein the filler is selected from the group consisting of talc, mica, silica, kaolin, polyamide powders, poly-β-alanine powders, polyethylene powders, tetrafluoroethylene polymers powder, lauroyllysine, starch, boron nitride, hollow polymer microspheres, acrylic polymer particles, silicone resin microbeads, precipitated calcium carbonate, dicalcium phosphate, magnesium carbonate, magnesium hydrocarbonate, hydroxyapatite, hollow silica microspheres, glass or ceramic microcapsules, organic carboxylic acids metal soaps containing from 8 to 22 carbon atoms; organic carboxylic acids metal soaps containing from 12 to 18 carbon atoms, and mixtures thereof.

33. The product as claimed in claim 30, wherein the filler is in an amount of from 0.1% to 35% by weight of the composition.

34. The product as claimed in claim 30, wherein the filler is in an amount of from 0.5% to 25% by weight of the composition.

35. The product as claimed in claim 30, wherein the filler is in an amount of from 1% to 15% by weight of the first composition.

36. The product as claimed in claim 1, wherein the physiologically acceptable medium in the second composition comprises a liquid phase that is nonvolatile at room temperature and atmospheric pressure.

37. The product as claimed in claim 36, wherein the nonvolatile liquid phase comprises one or more of a liquid hydrocarbon-based phase, a liquid fluoro phase and a liquid silicone phase.

38. The product as claimed in claim 36, wherein the nonvolatile liquid phase comprises a hydrocarbon-based phase with solubility parameters δD and δa according to the Hansen solubility space satisfying the following conditions:
8≦δD≦22 (J/cm³)^{½}, and 7≦δa≦35 (J/cm³)^{½}.

39. The product as claimed in claim 38, wherein 12≦δD≦19 (J/cm³) ^{½}.

40. The product as claimed in claim 38, wherein 16≦δD≦19 (J/cm³) ^{½}.

41. The product as claimed in claim 38, wherein 8≦δa≦20 (j/cm³) ^{½}.

42. The product as claimed in claim 38, wherein 8.5≦δa≦12 (J/cm³).

43. The product as claimed in claims 37, wherein the hydrocarbon-based phase comprises at least one hydrocarbon compound selected from the group consisting of diisostearyl malate, octyldodecanol, propylene glycol monoisostearate, polyglyceryl-2 diisostearate, castor oil, polyglyceryl-3 diisostearate, polyglyceryl-2 isostearate, butylene glycol, propylene glycol, glycerol, and mixtures thereof.

44. The product as claimed in claim 36, wherein the fluoro phase comprises one or more of fluorosilicone compounds, fluoro polyethers, and fluoroalkanes.

45. The product as claimed in claim 44, wherein the fluorosilicone compound has the formula (I): 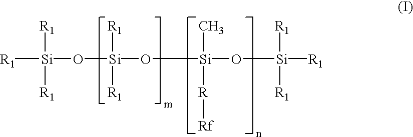
wherein
R is a linear or branched divalent alkyl group containing 1 to 6 carbon atoms;
Rf is a fluoroalkyl radical containing 1 to 9 carbon atoms;
R₁ are independently a C₁-C₂₀ alkyl radical, a hydroxyl radical or a phenyl radical;
m is from 0 to 150; and
n is from 1 to 300.

46. The product as claimed in claim 45, wherein R is a linear or branched divalent methyl, ethyl, propyl or butyl group.

47. The product as claimed in claim 45, wherein Rf is a perfluoroalkyl radical.

48. The product as claimed in claim 45, wherein the fluoroalkyl radical contains 1 to 4 carbon atoms.

49. The product as claimed in claim 45, wherein m is from 20 to 100.

50. The product as claimed in claim 45, wherein n is from 1 to 100.

51. The product as claimed in claim 44, wherein the fluoro polyether is a compound of formula (II):
R₆—(CF₂—CFR₃—CF₂O)p— (CFR₄—CF₂—O)q—(CFR₅—O)r—R₇ (II)
wherein
R₃, R₄, R₅, and R₆ are independently a monovalent radical selected from the group consisting of —F, —(CF₂)n—CF₃ and —O—(CF₂)n—CF₃;
R₇ is a monovalent —F radical or a monovalent —(CF₂)n—CF₃ radical;
n is from 0 to 4;
p is from 0 to 600;
q is from 0 to 860;
r is from 0 to 1,500, and wherein p, q and r are integers chosen such that the weight-average molecular weight of the compound 1s from 500 to 100,000.

52. The product as claimed in claimed 50, wherein p, q, and r are integers chosen such that the weight-average molecular weight of the compounds 1s from 500 to 10,000.

53. The product as claimed in claim 44, wherein the fluoroalkanes are selected from the group consisting of C₂-C₅₀ perfluoroalkanes, C₂-C₅₀ fluoroalkanes, perfluoroadamantane, bromoperfluorooctyl, and mixtures thereof.

54. The product as claimed in claim 53, wherein the fluoroalakanes are C₅-C₃₀ perfluoroalkanes or C₅-C₃₀ fluoroalkanes.

55. The product as claimed in claim 53, wherein the fluoroalkane is perfluorodecalin.

56. The product as claimed in claim 36, wherein the nonvolatile liquid phase comprises a silicone phase with at least one silicone oil.

57. The product as claimed in claim 56, wherein the silicone oil is phenylsilicone oil with a viscosity of from 5 to 100,000 cSt at 25° C.

58. The product as claimed in claim 56, wherein the silicone oil is phenylsilicone oil with a viscosity of from 5 to 10 000 cSt at 25° C.

59. The product as claimed in claim 57, wherein the phenylsilicone oil has the formula (A): 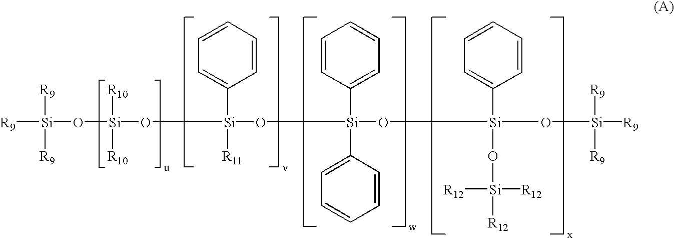
wherein
R₉ and R₁₂ are independently a C₁-C₃₀ alkyl radical, an aryl radical or an aralkyl radical;
R₁₀ and R₁₁ are independently a C₁-C₃₀ alkyl radical or an aralkyl radical; and
u, v, w and x are independently integers of from 0 to 900; wherein the sum v+w+x is not 0 and the sum u+v+w+x is from 1 to 900.

60. The product as claimed in claim 36, wherein the nonvolatile liquid phase comprises a silicone phase with a low-viscosity phenylsilicone oil and a high-viscosity phenylsilicone oil.

61. The product as claimed in claim 60, wherein the low-viscosity phenylsilicone oil has a viscosity of from 5 to 499 cSt at 25° C. and the high-viscosity phenylsilicone oil has a viscosity of from 500 to 10,000 cSt at 25° C.

62. The product as claimed in claim 60, wherein the low-viscosity phenylsilicone oil has a viscosity of from 5 to 300 cSt at 25° C., and the high-viscosity phenylsilicone oil has a viscosity of from 600 to 5,000 cSt at 25° C.

63. The product as claimed in claim 60, wherein the low-viscosity phenylsilicone oil has the formula (III): 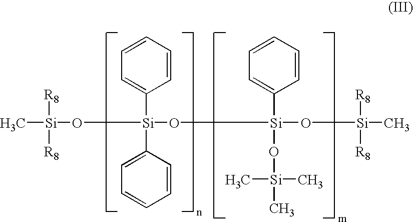
wherein
R₈ is a C₁-C₃₀ alkyl radical, an aryl radical or an aralkyl radical,
n is an integer of from 0 to 100;
m is an integer of from 0 to 100, wherein the sum of m+n is from 1 to 100.

64. The product as claimed in claim 60, wherein the weight ratio of the low-viscosity phenylsilicone oil and the high-viscosity phenylsilicone oil is from 70/30 to 30/70.

65. The product as claimed in claim 60, wherein the weight ratio of the low-viscosity phenylsilicone oil and the high-viscosity phenylsilicone oil is from 60/40 to 40/60.

66. The product as claimed in claim 60, wherein the weight ratio of the low-viscosity phenylsilicone oil and the high-viscosity phenylsilicone oil is from 55/45 to 45/55.

67. The product as claimed in claim 36, wherein the nonvolatile liquid phase is in an amount of from 1% to 100% of the total weight of the second composition.

68. The product as claimed in claim 36, wherein the nonvolatile liquid phase is in an amount of from 5% to 95% of the total weight of the second composition.

69. The product as claimed in claim 36, wherein the nonvolatile liquid phase is in an amount of from 20% to 80% of the total weight of the second composition.

70. The product as claimed in claim 36, wherein the nonvolatile liquid phase is in an amount of from 40% to 80% of the total weight of the second composition.

71. The product as claimed in claim 1, wherein the goniochromatic coloring agent is a mesomorphic coloring agent, a liquid-crystal coloring agent, or a multilayer interference structure.

72. The product as claimed in claim 1, wherein the goniochromatic coloring agent is a linear polymer with a grafted mesomorphic group, or cyclic polymer with a grafted mesomorphic group.

73. The product as claimed in claim 1, wherein the goniochromatic coloring agent is a silicone polymer or a cellulose ether with grafted cholesteric and biphenyl groups.

74. The product as claimed in claim 1, wherein the goniochromatic coloring agent is a cyclic silicone with polymerized monomers, wherein at least one monomer has the formula (IV): 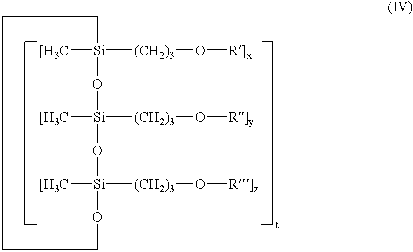
wherein
0≦x≦1;
0≦y≦1;
0≦z≦1; wherein x+y+z is not 0;
3≦t≦10;
R′ is a group of the following formula: 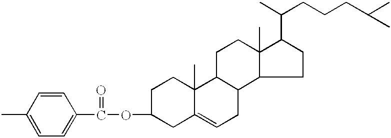
R″ is a group of the following formula: 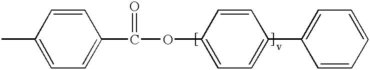
R″′ is a group of the following formula: 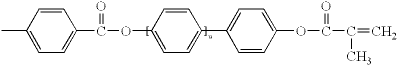
wherein u and v are independently 0 or 1; and
one or more monomers selected from the group consisting of cholesteryl methacrylate, cholesteryl 4-(2-propen-1-oxy)benzoate, and 4-methacryloyloxyphenyl 4-(2-propen-1-oxy)benzoate.

75. The product as claimed in claim 74, wherein x is 1.

76. The product as claimed in claim 74, wherein y is 1.

77. The product as claimed in claim 74, wherein z is 1.

78. The product as claimed in claim 1, wherein the goniochromatic agent comprises a multilayer interference structure selected from the group consisting of
Al/SiO₂/Al/SiO₂/Al;
Cr/MgF₂/Al/MgF₂/Al;
MOS₂/SiO₂/Al/SiO₂/MOS₂;
Fe₂O₃/SiO₂/Al/SiO₂/Fe₂O₃;
Fe₂O₃/SiO₂/Fe₂O₃/SiO₂/Fe₂O₃;
MOS₂/SiO₂/mica-oxide/SiO₂/MOS₂; and
Fe₂O₃/SiO₂/mica-oxide/SiO₂/Fe₂O₃.

79. The product as claimed in claim 1, wherein the monochrome coloring agent is a liposoluble monochrome dye, a water-soluble monochromic dye, a monochromic pigment, or a nacre.

80. The product as claimed in claim 1, wherein the monochromic coloring agent exhibits one of the colors of the goniochromatic agent.

81. The product as claimed in claim 1, wherein the monochromic coloring agent is selected from the group consisting of titanium oxide, zirconium oxide cerium oxide, zinc oxide, iron oxide, chromium oxide, ferric blue, carbon black, barium lakes, strontium lakes, calcium lakes, aluminum lakes, Sudan red, DC Red 17, DC Green 6, β-carotene, soybean oil, Sudan brown, DC Yellow 11, DC Violet 2, DC Orange 5, quinoline yellow, annatto, mica coated with titanium oxide, mica coated with iron oxide, mica coated with natural pigment, mica coated with bismuth oxychloride, and mixtures thereof.

82. The product as claimed in claim 1, wherein the first coloring agent is in amount of from 0.001% to 60% of the total weight of the first composition; and the second coloring agent is in an amount of from 0.001% to 60% of the total weight of the second composition.

83. The product as claimed in claim 1, wherein the first coloring agent is in an amount of from 0.01% to 50% of the total weight of the first composition; and the second coloring agent is in an amount of from 0.01% to 50% of the total weight of the second composition.

84. The product as claimed in claim 1, wherein the first coloring agent is in an amount of from 0.1% to 40% of the total weight of the first composition; and the second coloring agent is in an amount of from 0.1% to 40% of the total weight of the second composition.

85. The product as claimed in claim 1, wherein the first coloring agent is in a dispersion.

86. The product as claimed in claim 1, wherein the second coloring agents are in a dispersion.

87. The product as claimed in claim 1, wherein the physiologically acceptable medium for the first aor the second composition comprises one or more cosmetic or dermatological active agents.

88. The product as claimed in claim 1, wherein the physiologically acceptable medium for the first and/or second composition further comprises fatty compounds selected from the group consisting of waxes, oils, gums, hydrocarbon-based pasty fatty compounds, silicone-based fatty compounds, fluoro based fatty compounds, plant fatty compounds, animal fatty compounds, mineral fatty compounds, synthetic fatty compounds, and mixtures thereof.

89. The product as claimed in claim 1, wherein the physiologically acceptable medium in the first composition comprises a gum.

90. The product as claimed in claim 89, wherein the gum is a silicone gum.

91. The product as claimed in claim 1, wherein the physiologically acceptable medium in the second composition comprises one or both of a pasty fatty substance and a wax.

92. The product as claimed in claim 1, wherein the physiologically acceptable medium for the first and/or second composition further comprises at least one additive selected from the group consisting of oil thickeners, aqueous-phase thickeners, antioxidants, fragrances, preserving agents, surfactants, liposoluble polymers, and mixtures thereof.

93. The product as claimed in claim 1, wherein the first composition or second composition is an oily solution, an aqueous solution, an oily gel, an aqueous gel, an oil-in-water emulsion, a water-in-oil emulsion, a multiple emulsion, a dispersion of oil in water with vesicles, or a powder.

94. The product as claimed claim 1, wherein the first composition or second composition is anhydrous.

95. The product as claimed in claim 1, which is a foundation, a face powder, an eyeshadow, a lipstick, a colored lipcare product, an eyeliner, a concealer product or a body make-up product.

96. The product as claimed in claim 1, which is a lipstick.

97. The product as claimed in claim 1, wherein the second composition is solid.

98. The product as claimed in claim 1, further comprising a device for applying the first and second compositions to the skin, the lips, and/or integuments.

99. The product as claimed in claim 98, wherein the device is selected from the group consisting of a fine brush, a coarse brush, a pen, a pencil, a felt, a nib, a sponge and a foam.

100. The product as claimed in claim 99, wherein the device is a felt.

101. The product as claimed in claim 1, wherein the first and second compositions are packaged separately.

102. A process for making-up the skin, the lips and/or integuments, comprising
applying to the skin, the lips and/or integuments a coat of a first composition which contains polymer particles dispersed in a first physiologically acceptable medium, with a surface stabilized by a stabilizer in a liquid phase and at least one first coloring agent, and
applying, over all or a part of said first composition, a coat of a second composition which contains, in a second physiologically acceptable medium, at least two second coloring agents, wherein at least one of the second coloring agents is a goniochromatic agent that produces different colors under different light, and wherein at least one second coloring agent is a monochromic agent.

103. The process as claimed in claim 102, wherein the coat of the first composition is allowed to dry before applying the coat of the second composition.

104. The process as claimed in claim 102, wherein the second coat is discontinuous.

105. The process as claimed in claim 102, wherein the second coat is applied in a pattern.

106. The process as claimed in claim 102, wherein the physiologically acceptable medium in the second composition comprises a liquid phase that is nonvolatile at room temperature and atmospheric pressure.

107. The process as claimed in claim 106, wherein the nonvolatile liquid phase comprises at least one of a liquid hydrocarbon-based phase, a liquid fluoro phase or a liquid silicone phase.

108. The process as claimed in claim 106, wherein the nonvolatile liquid phase comprises a hydrocarbon-based phase with solubility parameters δD and δa according to the Hansen solubility space satisfying the following conditions: 8≦δD≦22 (J/cm³)^{½}, and 7≦δa≦35 (J/cm³)^{½}.

109. The process as claimed in claim 108, wherein 12≦δD≦19 (J/cm³)^{½}.

110. The process as claimed in claim 108, wherein 16≦δD≦19 (J/cm³)^{½}.

111. The process as claimed in claim 108, wherein 8≦δa≦20 (J/cm³)^{½}.

112. The process as claimed in claim 108, wherein 8.5≦δa≦12 (J/cm³)^{½}.

113. The process as claimed in claim 107, wherein the hydrocarbon-based phase comprises at least one of diisostearyl malate, octyldodecanol, propylene glycol monoisostearate, polyglyceryl-2 diisostearate, castor oil, polyglyceryl-3 diisostearate, polyglyceryl-2 isostearate, butylene glycol, propylene glycol, glycerol, or mixtures thereof.

114. The process as claimed in claim 107, wherein the nonvolatile liquid phase comprises the fluoro phase which contains at least one of a fluorosilicone compound, a fluoro polyether, or a fluoroalkane.

115. The process as claimed in claim 114, wherein the fluorosilicone compound has the formula (I): 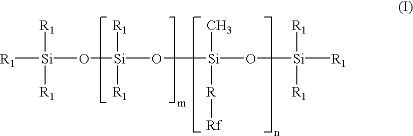
wherein
R is a linear or branched divalent alkyl group containing 1 to 6 carbon atoms;
Rf is a fluoroalkyl radical containing 1 to 9 carbon atoms;
R₁ are independently a C₁-C₂₀ alkyl radical, a hydroxyl radical or a phenyl radical;
m is from 0 to 150; and
n is from 1 to 300.

116. The process as claimed in claim 115, wherein R is a linear or branched divalent methyl, ethyl, propyl or butyl group.

117. The process as claimed in claim 115, wherein Rf is a perfluoroalkyl radical.

118. The process as claimed in claim 115, wherein the fluoroalkyl radical contains 1 to 4 carbon atoms.

119. The process as claimed in claim 115, wherein m is from 20 to 100.

120. The process as claimed in claim 115, wherein n is from 1 to 100.

121. The process as claimed in claim 114, wherein the fluoro polyether is a compound of formula (II):
R₆—(CF₂—CFR₃—CF₂O)p—(CFR₄—CF₂—O)q—(CFR₅—O)r—R₇ (II)
wherein
R₃, R₄, R₅, and R₆ are independently a monovalent radical selected from the group consisting of —F, —(CF₂)n—CF₃ and —O—(CF₂)n—CF₃;
R₇ is a monovalent —F radical or a monovalent —(CF₂)n—CF₃ radical;
n is from 0 to 4;
p is from 0 to 600;
q is from 0 to 860;
r is from 0 to 1,500, and wherein p, q and r are integers chosen such that the weight-average molecular weight of the compound 1s from 500 to 100,000.

122. The process as claimed in claimed 121, wherein p, q, and r are integers chosen such that the weight-average molecular weight of the compounds 1s from 500 to 10,000.

123. The process as claimed in claim 114, wherein the fluoroalkanes are selected from the group consisting of C₂-C₅₀ perfluoroalkanes, C₂-C₅₀ fluoroalkanes, perfluoroadamantane, bromoperfluorooctyl, and mixtures thereof.

124. The process as claimed in claim 106, wherein the nonvolatile liquid phase comprises a silicone phase with at least one silicone oil.

125. The process as claimed in claim 124, wherein the silicone oil is a phenylsilicone oil with a viscosity of from 5 to 100,000 cSt 25° C.

126. The process as claimed in claim 124, wherein the silicone oil is a phenylsilicone oil with a viscosity of from 5 to 10,000 cSt at 25° C.

127. The process as claimed in claim 124, wherein the silicone oil is a phenylsilicone oils with the formula (A): 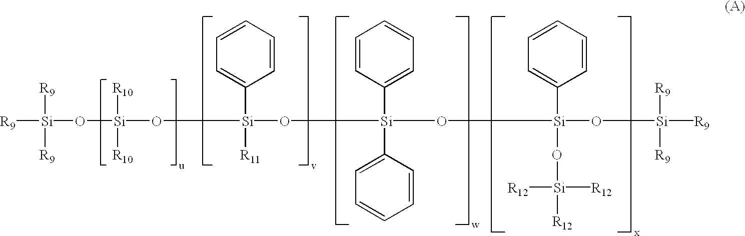
wherein
R₉ and R₁₂ are independently a C₁-C₃₀ alkyl radical, an aryl radical or an aralkyl radical;
R₁₀ and R11 are independently a C₁-C₃₀ alkyl radical or an aralkyl radical;
u, v, w and x are independently integers from 0 to 900; wherein the sum v+w+x is not 0 and that the sum u+v+w+x is from 1 to 900.

128. The process as claimed in claim 106, wherein the nonvolatile liquid phase comprises a silicone-based phase with at least a low-viscosity phenylsilicone oil and a high-viscosity phenylsilicone oil.

129. The process as claimed in claim 128, wherein the low-viscosity phenylsilicone oil has a viscosity of from 5 to 499 cSt at 25° C and the high-viscosity phenylsilicone oil has a viscosity of from 500 to 10,000 cSt at 25° C.

130. The process as claimed in claim 128, wherein the low-viscosity phenylsilicone oil has a viscosity of from 5 to 300 cSt at 25° C., and the high-viscosity phenylsilicone oil has a viscosity of from 600 to 5,000 cSt at 25° C.

131. The process as claimed in claim 128, wherein the low-viscosity phenylsilicone oil has the formula (III): 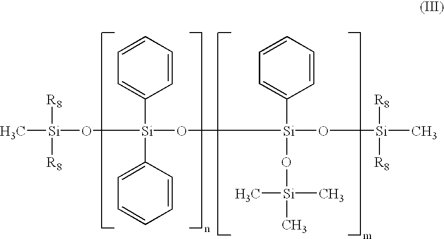
wherein
R₈ is a C₁-C₃₀ alkyl radical, an aryl radical or an aralkyl radical;
n is an integer of from 0 to 100; and
m is an integer of from 0 to 100; wherein the sum m+n is from 1 to 100.

132. The process as claimed in claim 128, wherein the weight ratio of the low-viscosity phenylsilicone oil and the high-viscosity phenylsilicone oil is from 70/30 to 30/70.

133. The process as claimed in claim 128, wherein the weight ratio of the low-viscosity phenylsilicone oil and the high-viscosity phenylsilicone oil is from 60/40 to 40/60.

134. The process as claimed in claim 128, wherein the weight ratio of the low-viscosity phenylsilicone oil and the high-viscosity phenylsilicone oil is from 55/45 to 45/55.

135. The process as claimed in claim 106, wherein the nonvolatile liquid phase is in an amount of from 1% to 100% of the total weight of the second composition.

136. The process as claimed in claim 106, wherein the nonvolatile liquid phase is in an amount of from 5% to 95% of the total weight of the second composition.

137. The process as claimed in claim 106, wherein the nonvolatile liquid phase is in an amount of from 20% to 80% of the total weight of the second composition.

138. The process as claimed in claim 106, wherein the nonvolatile liquid phase is in an amount of from 40% to 80% of the total weight of the second composition.

139. The process as claimed in claim 102, wherein the goniochromatic coloring agent is a mesomorphic coloring agent, a liquid-crystal coloring agents, or a multilayer interference structure.

140. Thee process as claimed in claim 102, wherein the goniochromatic coloring agent is a linear polymer with a grafted mesomorphic group or cyclic polymer with a grafted mesomorphic group.

141. The process as claimed in claim 102, wherein the goniochromatic coloring agent is a silicone polymer or a cellulose ether with grafted cholesteric and biphenyl groups.

142. The process as claimed in claim 102, wherein the goniochromatic coloring agent is a cyclic silicone with polymerized monomers, wherein at least one monomer having the formula: 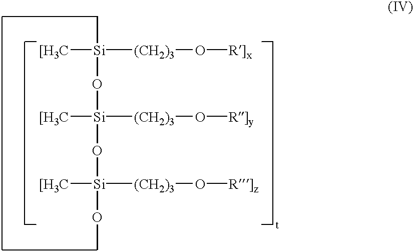
wherein
0≦x≦1; 0≦y≦1; 0≦z≦1; wherein x+y+z is not 0;
3≦t≦10;
R′ is a group of the following formula: 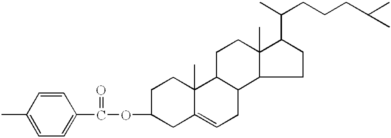
R″ is a group of the following formula: 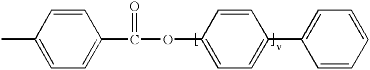
R″′ is a group of the following formula: 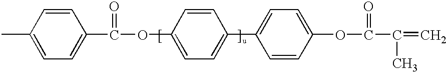
wherein u and v are independently 0 or 1, and one or more monomers selected from the group consisting of cholesteryl methacrylate, cholesteryl 4-(2-propen-1-oxy)benzoate, and 4-methacryloyloxyphenyl 4-(2-propen-1-oxy)benzoate.

143. The process as claimed in claim 142, wherein x is 1.

144. The process as claimed in claim 142, wherein y is 1.

145. The process as claimed in claim 142, wherein z is 1.

146. The process as claimed in claim 142, wherein the goniochromatic coloring agent comprises a multilayer interference structure selected from the group consisting of
Al/SiO₂/Al/SiO₂/Al;
Cr/MgF₂/Al/MgF₂/Al;
MOS₂/SiO₂/Al/SiO₂/MOS₂;
Fe₂O₃/SiO₂/Al/Sio₂/Fe₂O₃;
Fe₂O₃/SiO₂/Fe₂O₃/SiO₂/Fe₂O₃;
MOS₂/SiO₂/mica-oxide/SiO₂/MOS₂; and
Fe₂O₃/SiO₂/mica-oxide/SiO₂/Fe₂O₃.

147. The process as claimed in claim 102, wherein the monochromic coloring agent is a liposoluble monochromic dye, a water-soluble monochromic dye, a monochromic pigment or nacres.

148. The process as claimed in claim 102, wherein the monochromic coloring agent exhibits one of the colors of the goniochromatic coloring agent.

149. The process as claimed in claim 102, wherein the first coloring agent is in amount of from 0.001% to 60% of the total weight of the first composition; and the second coloring agent is in an amount of from 0.001% to 60% of the total weight of the second composition.

150. The process as claimed in claim 102, wherein the first coloring agent is in an amount of from 0.01% to 50% of the total weight of the first composition; and the second coloring agent is in an amount of from 0.01% to 50% of the total weight of the second composition.

151. The process as claimed in claim 102, wherein the first coloring agent is in an amount of from 0.1% to 40% of the total weight of the first composition; and the second coloring agent is in an amount of from 0.1% to 40% of the total weight of the second composition.

152. The process as claimed in claim 102, wherein the first coloring agent is in a dispersion.

153. The process as claimed in claim 102, wherein the second coloring agent is in a dispersion.

154. The process as claimed in claim 102, wherein the monochromic coloring agent is selected from the group consisting of titanium oxide, zirconium oxide cerium oxide, zinc oxide, iron oxide, chromium oxide, ferric blue, carbon black and barium, strontium, calcium or aluminum lakes, Sudan red, DC Red 17, DC Green 6, β-carotene, soybean oil, Sudan brown, DC Yellow 11, DC Violet 2, DC Orange 5, quinoline yellow, annatto, mica coated with titanium oxide, mica coated with iron oxide, mica coated with natural pigment, mica coated with bismuth oxychloride, and mixtures thereof.

155. A cosmetic composition comprising, polymer particles dispersed in a physiologically acceptable medium, which is surface-stabilized by a stabilizer in a liquid phase;
a coloring agent that is a goniochromatic coloring agent that produces different colors under different light and the angle of observation, or monochrome coloring agent; and
a rheological agent, which is an olefin copolymer of controlled crystallization.

156. The composition as claimed in claim 155, wherein the rheological agent is an ethylene/octene copolymer.

157. An article comprising
a coat of a first composition, which contains polymer particles dispersed in a first physiologically acceptable medium, which is surface stabilized by a stabilizer in a liquid phase; and at least one first coloring agent; and
a coat of a second composition, applied over all or a part of the first coat, which contains, in a second physiologically acceptable medium, at least two second coloring agents, wherein at least one of the coloring agents is a goniochromatic agent that produces different colors under different light and the angle of observation, and at least one of the coloring agents is a monochromic agent; wherein the second composition is in a solid form.

158 The article as claimed in claim 157, which is in a form selected from the group consisting of a false nail, a false eyelash, and a wig.
